(19) United States
(12) Patent Application Publication
(10) Pub. No.: US 2022/0249323 A1
(43) Pub. Date: Aug. 11, 2022

(54) METHOD OF NON-INVASIVE FIGURE CORRECTION AND METHOD OF MASSAGE FOR NON-INVASIVE FIGURE CORRECTION

(71) Applicant: Anna Aleksandrovna KALICHKINA, G. Strezhevoy, Tomskaya oblast (RU)

(72) Inventor: Anna Aleksandrovna KALICHKINA, G. Strezhevoy, Tomskaya oblast (RU)

(21) Appl. No.: 17/630,316

(22) PCT Filed: Jun. 2, 2020
(86) PCT No.: PCT/RU2020/050108

§ 371 Date: Jan. 26, 2022

(30) Foreign Application Priority Data
Aug. 7, 2019 (RU) 2019 124999

(51) Int. Cl. A61H 99/00

(52) U.S. Cl. CPC A61H 99/00; A61H 2205/083; A61H 2203/0456

(57) ABSTRACT

A method for non-surgical body sculpting is claimed, characterized by the compilation of a set of measures for body sculpting, including cleansing the body; regulating water metabolism, diet and rest; unloading the gastrointestinal tract using mono-days and improving nutrition, taking vitamins and completely eliminating alcohol and foods that are digested in the stomach more than 2 hours, eating in portions of 150-200 ml, 5-6 times a day; physical exercises aimed at forming a healthy posture, tactile techniques of impact, while the muscles in the affected areas are pre-stimulated to improve blood circulation through physical exercises. A method of massage for non-surgical body sculpting, which includes the impact on fat deposits in the abdomen, peritoneum and weakened abdominal muscles in the form of a combination of physical activity and massage.

### FIELD OF INVENTION

The invention relates to methods for drug-free health improvement of a person, and may be used when carrying out medical-recreational and health promotion measures, in the field of physical culture and medicine, namely in dietetics, cosmetology.

### BACKGROUND OF THE INVENTION

Functional disorders of the body associated with a violation of diet and fluid intake, metabolic disorders and insufficient physical activity are widespread at present. Overweight, which is often a consequence of these disorders, is recognized as a disease by the World Health Organization. According to the WHO data for 2016, 39% of adults over 18 years old (39% of men and 40% of women) were overweight, about 13% of the world's adult population (11% of men and 15% of women) were obese. An increased body mass index (BMI) is one of the main risk factors for non-communicable diseases such as cardiovascular diseases, diabetes, disorders of the musculoskeletal system (especially osteoarthritis, an extremely disabling degenerative joint disease), some cancers (including cancer of the endometrium, breast, ovary, prostate, liver, gallbladder, kidney and colon); the risk of these non-communicable diseases grows as the BMI increases. And, like any disease, overweight requires both treatment and prevention. Overweight and obesity, as well as related non-communicable diseases, are largely preventable, according to the WHO. The WHO cites energy imbalances as the main causes of obesity and overweight, in which the caloric content of the diet exceeds the energy requirements of the body, while pointing out that worldwide there is an increase in the consumption of foods with high energy density and high fat content and a decrease in physical activity due to an increasingly sedentary nature of many activities, changes in travel methods and increasing urbanization. A transition to a healthier diet and regular physical activity plays a critical role in tackling overweight problems, which will help to prevent overweight and obesity. At the same time, it is important not only to reduce weight, but to qualitatively change the state of the body, to make it stronger, healthier and more balanced.

From the background art, complex methods of impact are widely known, aimed at reducing overweight and body sculpting. In particular, such methods are disclosed in patents RU2651080 Method for the Comprehensive Improvement of the Human Body (A61H 1/00, A61B 5/00, A61H 7/00, A61M 21/00, A61M 3/00, A61M 35/00, A61K 35/02, A61K 36/00, A61P 43/00, published on 18 Apr. 2018), RU2481846 METHOD FOR INTEGRATED RECOVERY OF THE HUMAN BODY IN THE PROCESS OF ECO-AUDIT AND ECO-MONITORING (IPC A61K 33/08, A61K 36/8962, A61K 36/87, A61K 36/28, A61K 36/55, A61P 3/00, published on 20 May 2013), RU2127574 METHOD FOR HUMAN HEALTH IMPROVEMENT (IPC A61H 1/00, A61H 1/02, published on 20 Mar. 1999), RU1102598 Method for Treating Obese Patients (IPC A61H 1/00, published on 15 Jul. 1984), CN108112992 Functional Medical Nutrition Weight Loss Formula and Method (IPC A23L29/00; A23L33/00; A23L33/10; A23L33/105; A23L33/15; A23L33/155; A23L33/16, published on May 6, 2018), CN106942730 Weight-Loss Formula and Method Capable of Healthily Clearing Body and Decomposing Fat (IPC A23L33/00; A23L33/12; A23L33/135; A23L33/15; A23L33/16; A23L33/21, published on 14 Jul. 2017), US20110196212 Methods and Systems for Health Wellness Management (IPC A61B5/00, published on Nov. 8, 2011).

As the closest analogue, characterized by the identification of problem areas and the preparation of a set of measures, including cleansing procedures, nutrition improvement, physical activity, breathing exercises, tactile, massage and instrumentation techniques, cold exposure training, the chosen solution is METHOD FOR RECOVERY OF THE HUMAN BODY according to patent RU2509547 (IPC A61H 7/00, A61H 33/00, A61H 99/00, A61B 8/00, A61B 10/00, A61K 33/00, A61K 36/899, A61P 1/16, A23L 1/29, published on 20 Mar. 2014).

In the prior art solution, much attention is paid to the cleansing of internal organs and various instrumentation effects to eliminate external manifestations of overweight, such as cellulite, or the consequences of weight loss in the form of flabby, sagging skin, while such effects do not allow to strengthen the corpus muscles to form a healthy posture and muscles, nor do they provide intense sweating that allows to remove more waste products and salts with sweat, thereby reducing the risk of kidney overload. This is very important, since with intense weight loss, most of the toxins from processed fat and other waste products are excreted through the liver and especially the kidneys.

### DISCLOSURE OF THE INVENTION

The problem addressed by the claimed solution is to create a comprehensive method of impacting the human body in order to reduce weight and sculpt the body, due to the general cleansing of the body from congestions, improving posture, and also due to the fact that excess body fat is removed from specific problem areas. The technical result consists in increasing the effectiveness of the impact of a set of measures aimed at weight loss and body sculpting. The claimed method can be implemented to sculpt the body of both women and men, of any age and state of health, even in the presence of chronic diseases.

The application task is solved and the claimed result is achieved in that the method of non-surgical body sculpting is characterized by the fact that they identify problem areas of the body, formulate a set of measures for body sculpting, including cleansing the excretory systems of the body; regulating water metabolism, diet and rest; unloading the gastrointestinal tract using mono-days and improving nutrition, taking vitamins and completely eliminating alcohol and foods whose digestion time in the stomach is more than 2 hours; eating in portions of 150-200 ml, 5-6 times a day; physical exercises aimed at forming a healthy posture, strengthening the corpus muscles and peritoneum, as well as working out problem areas; breathing exercises to enhance the burning of visceral fat, improve the supply of oxygen to the tissues of the body and perform self-massage of internal organs; tactile techniques of impact, while, immediately before carrying out procedures with tactile effects on certain areas, the muscles in these areas are preliminarily stimulated to improve blood circulation by means of physical exercises, before which warm-up is performed and intense sweating is started by alternating moderate and intense loads in phases of 30-60 seconds with heart rate and blood pressure regulation by breathing, followed by static strength exercises during breath holding, wherein water intake during physical exercises is excluded, but is required 1-1.5 hours before them. Tactile techniques of impact include, in particular, massage, instrumentation procedures, contrast showers, rubbing, body wrapping, and wearing compression underwear.

The claimed result is also achieved by using a massage method for non-surgical body sculpting, which includes the impact on fat deposits, the peritoneum and weakened abdominal muscles by way of a combination of physical activity and massage, in which, in the supine position with the straight legs brought together and the hands folded under the back of the head, a deep, soft expiration is produced from the abdomen, the abdomen is pulled in as much as possible, then, the area of connection of the abdominal muscles is fixed with the help of the operator, placing a hand in the middle of the abdomen: from the pubic bone (elbow) to the stomach area (wrist), and the abdomen is pressed; then, during breath holing, straightened legs are raised 30 cm from the surface, while raising the head, shoulders, shoulder-blades, tensing the abs and the muscles of the ribs; the abdomen is forcibly pressed with the operator's hand, not allowing the lower back to bend and the abdominal muscles to lift; with the knuckles of the operator's free hand, which is balled into fist, dynamic rubbing is carried out in the up and down direction along the abdominal muscles and the white line of the abdomen for a few seconds until the skin reddens; the cycle is repeated several times, alternating the impact on both sides.

The entire set of features of the claimed method contributes to a more effective impact of the measures applied on the process of weight reduction and retention, body sculpting, as well as on the health of the body as a whole.

Due to the fact that when compiling a set of measures aimed at reducing weight, sculpting the body and improving the body health, procedures are chosen that are designed to cleanse the body, in particular, the regulation of the urinary, lymphatic, circulatory and other systems, sweating, water metabolism; the functioning of all organs and metabolism are getting better, the process of weight reduction is faster, more efficient, safer in case of chronic diseases and with a longer retention of results.

As noted earlier, nutritional management plays a key role in losing weight and preventing an increase in BMI. To this end, the claimed method includes holding mono-days, taking foods, the digestion time of which is no more than 2 hours, eating in small portions of 150-200 ml (200-250 ml for men), 5-6 times a day, total abstinence from alcohol, as well as setting sleep and rest pattern, taking food and fluid in accordance with biorhythms. In the claimed solution, mono-days mean the kind of diet when only one dish or one type of food is consumed during the day. In addition, since the content of nutrients in food products is insufficient to fully cover the vitamins and minerals that the body needs, it is necessary to additionally take vitamins and food supplements to adjust the intake of the required amount of vitamins and mineral nutrients for the full functioning of all organs.

The claimed method also includes breathing exercises to improve the supply of oxygen to the tissues of the body, necessary for normal life. Oxygen is used by cells to oxidize organic matter with the release of energy expended in the process of vital activity (cellular or tissue respiration). In addition, breathing exercises can enhance the burning of visceral fat, strengthen the thin muscle of the peritoneum, intercostal muscles, abdominal muscles, back muscles and diaphragm, and performing physical exercises during breath holding in the claimed method stimulates increased sweating and metabolic processes, fat breakdown in the area where muscles work, and further, during inspiration, the maximum saturation of cells with oxygen.

The second cause of the appearance of overweight after an energy imbalance, under which the caloric content of the diet exceeds the energy requirements of the body, is a decrease in physical activity due to an increasingly sedentary lifestyle. In a large number of people, especially those suffering from obesity, the muscles that support the spine are often very weak, the lumbar region “falls” forward in hyperlordosis, which results in a bloated/hanging belly, sunken chest, and hunched back. To correct this cause, the claimed method includes physical exercises. In doing so, preference is given to general strengthening exercises aimed at strengthening the muscles of the body, joints, ligaments and spine in order to improve the tone and posture and to form a general fit, well-shaped appearance.

An important role in body sculpting while losing weight is played by various tactile, massage and instrumentation methods of impact, which allow one to first get rid of obvious superficial congestions, after which, when a deeper self-cleansing begins in the body, remove congestions from dense tissues, and then contribute to effective reduction of the fat layer, when it already changes its structure and is ready for active processing by the body and removal therefrom. This is when it is appropriate to proceed to intensive kneading of excess fat in order to correct the figure by focusing on specific problem areas. At the same time, for a more effective impact, immediately before exerting a tactile effect on certain problem areas, the muscles in these areas are pre-stimulated to improve blood circulation through physical exercises.

The peculiarity of performing physical exercises in the claimed method lies in the fact that before performing them, the maximum sweating is reached, while maintaining the heart rate and blood pressure indicators at average values, which is especially important for people with health problems that are often characteristic of overweight people. Good functioning of the sweat glands of the body allows to effectively control blood pressure. Intense sweating during a specific, moderate load on the muscles and the circulatory system allows to remove more waste products and salts with sweat, thereby reducing the risk of kidney overload. This is very important, because when reducing weight, most of the toxins from processed fat and other waste products are removed through the liver and especially the kidneys. Intense breathing makes it possible to remove as many waste products as possible with exhaled carbon dioxide, to saturate and supply body tissues with oxygen, which also enhances the processing of fat cells in tissues.

Before applying the claimed method or simultaneously with it, it is recommended to conduct a general examination of the body, to identify existing health problems in order to eliminate exacerbation of existing diseases through implementing a set of measures that stimulates the start of self-cleansing of the body and allows for correcting the figure in specific problem areas. The set of measures itself is also compiled on the basis of the analysis of the initial data obtained as a result of a preliminary examination, or from information according to the patient, concerning his/her state of health.

### BRIEF DESCRIPTION OF DRAWINGS

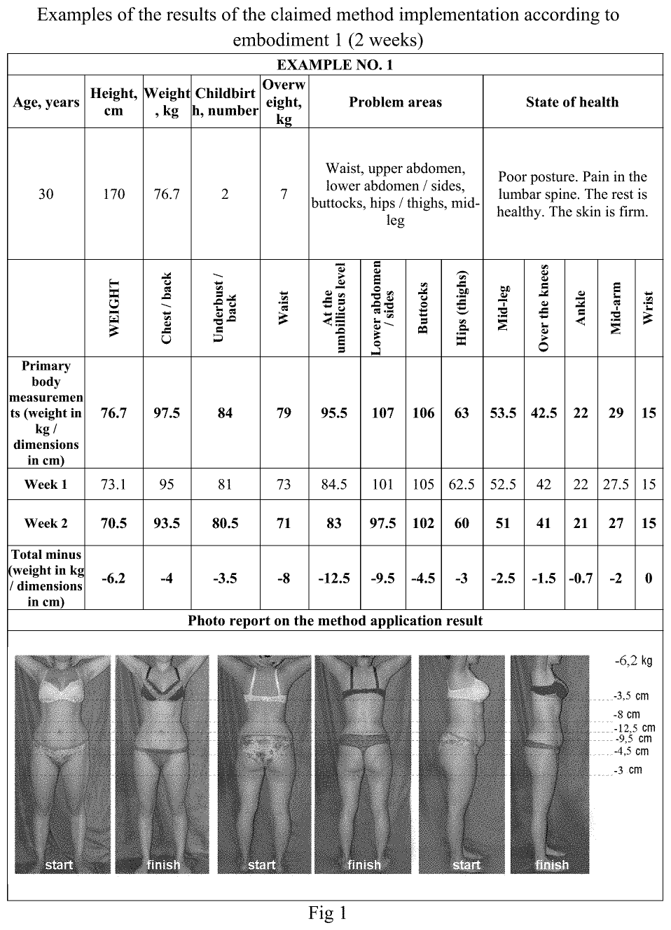

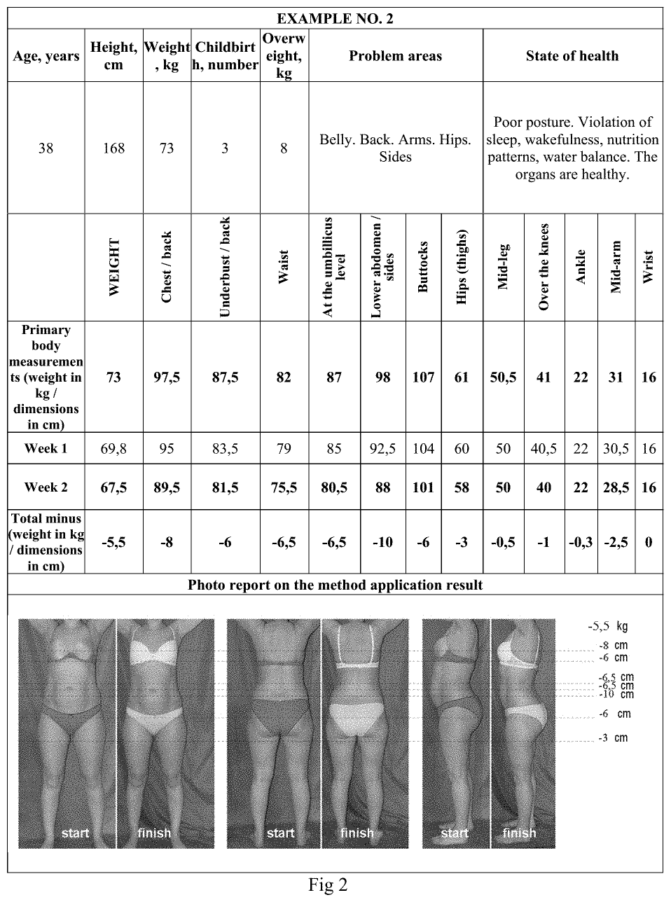

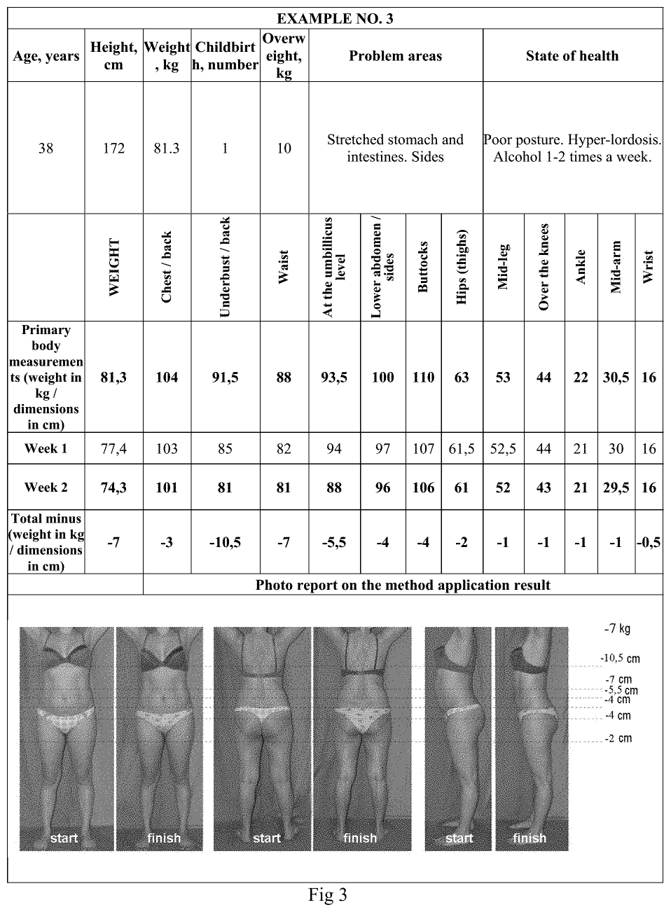

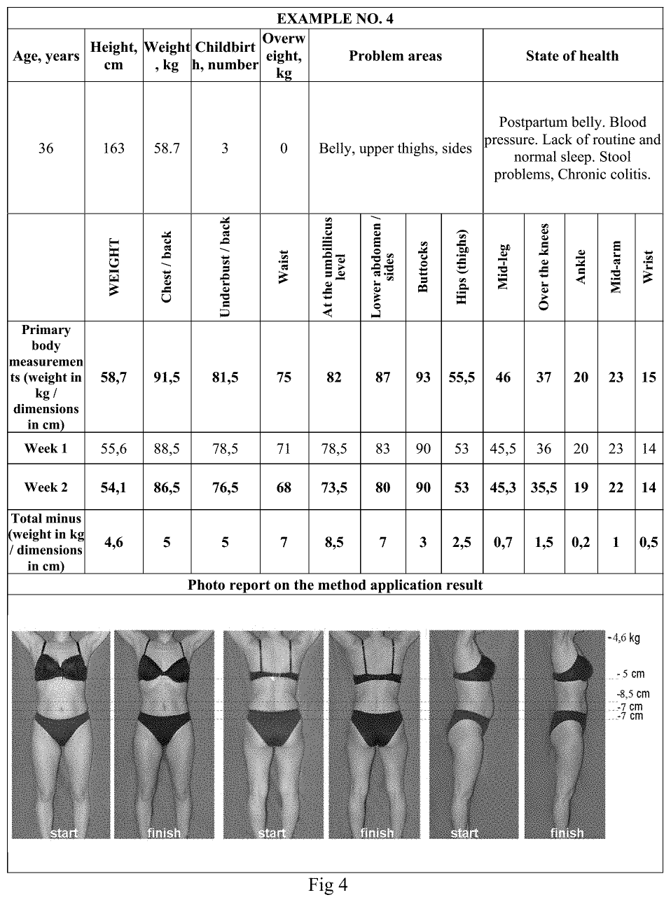

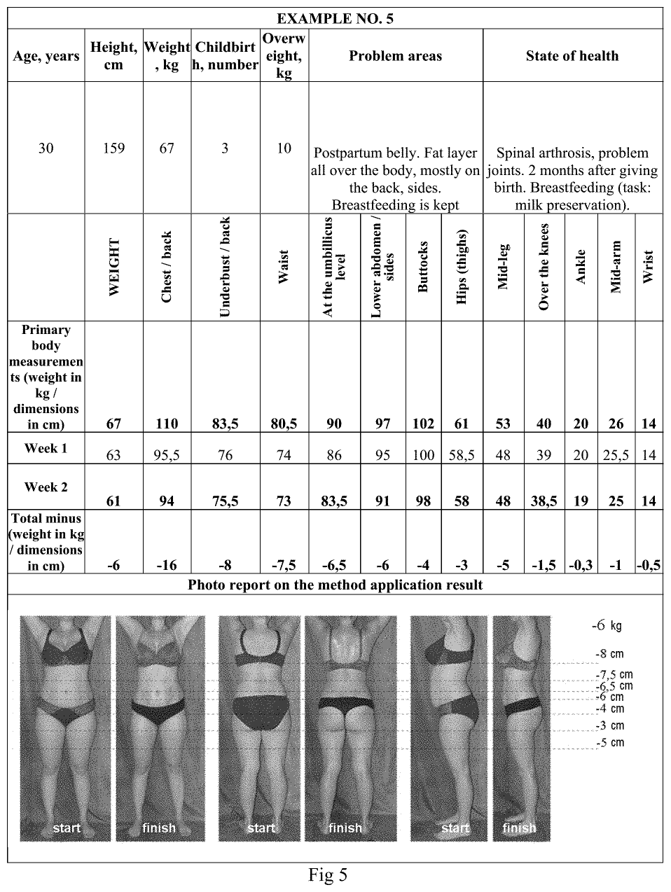

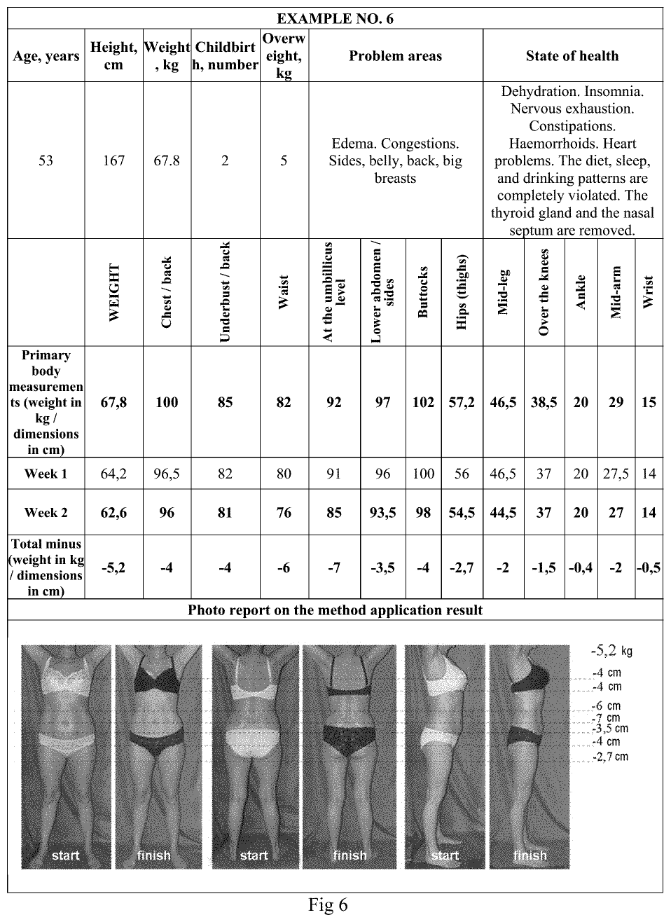

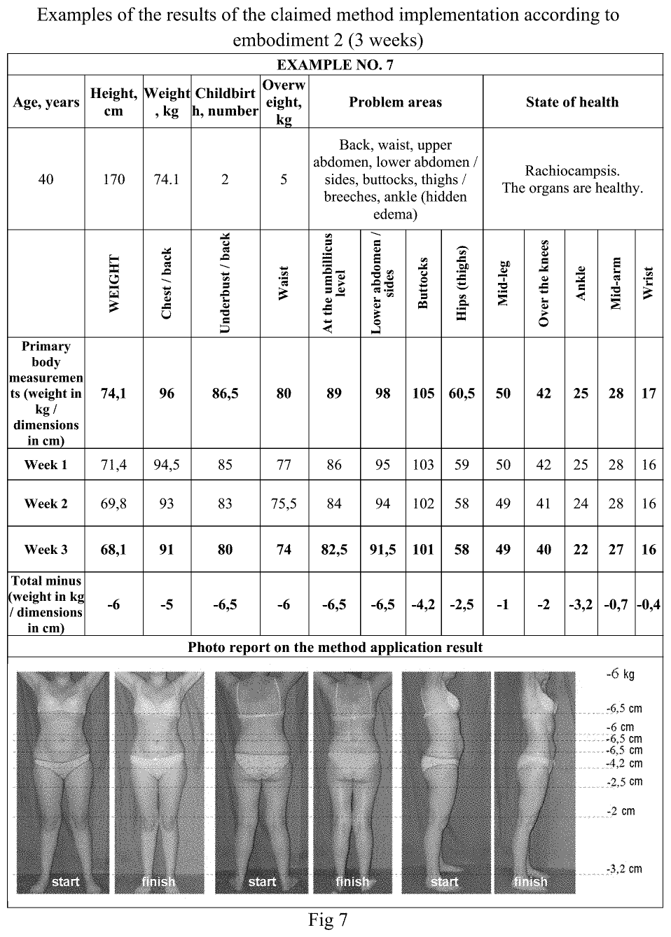

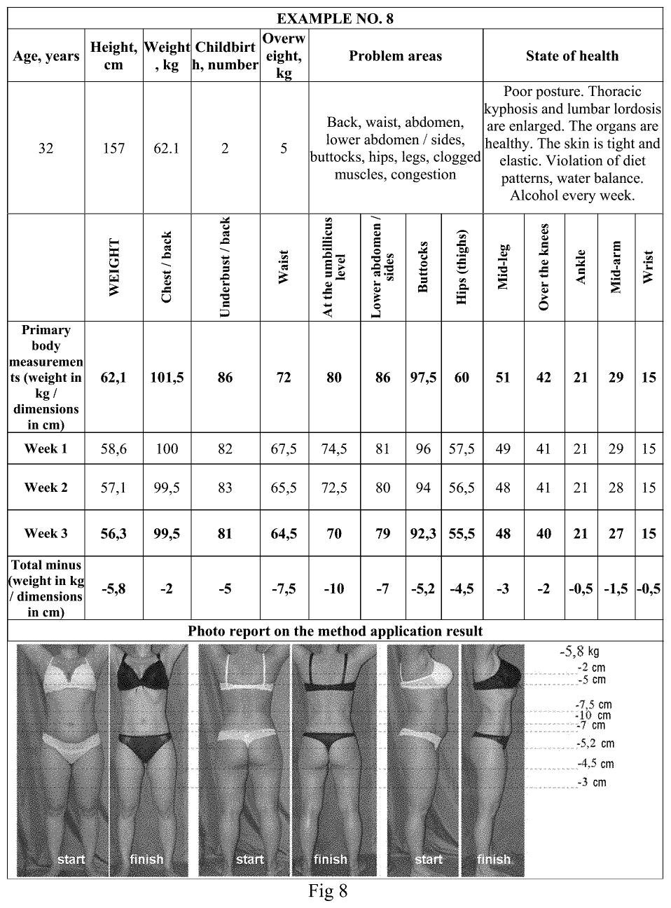

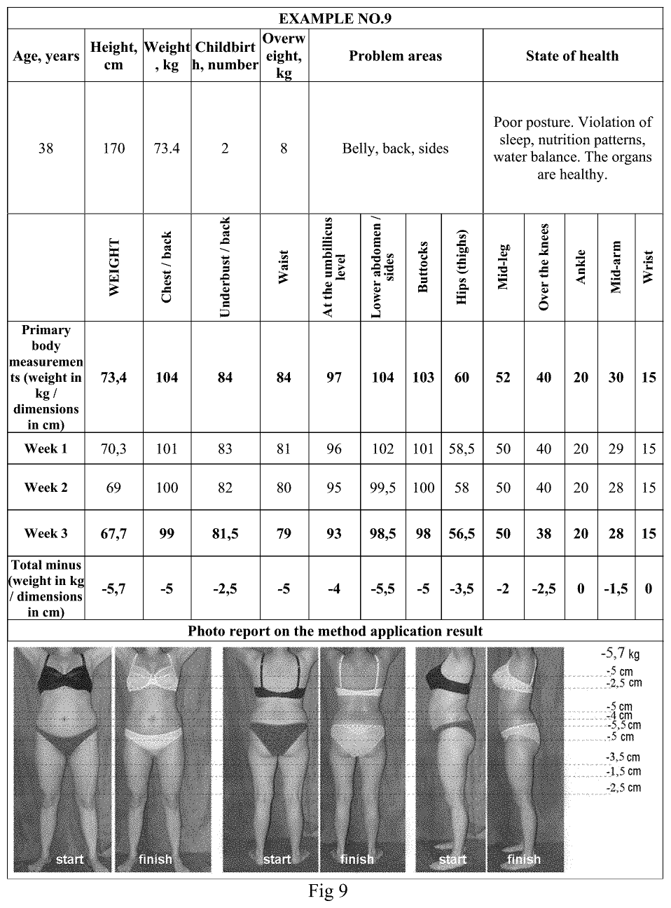

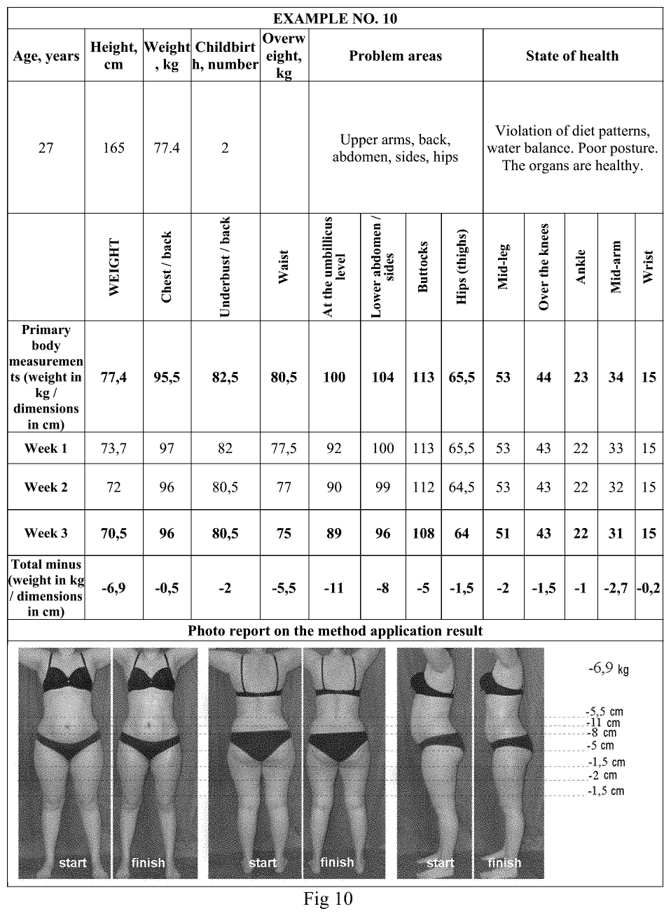

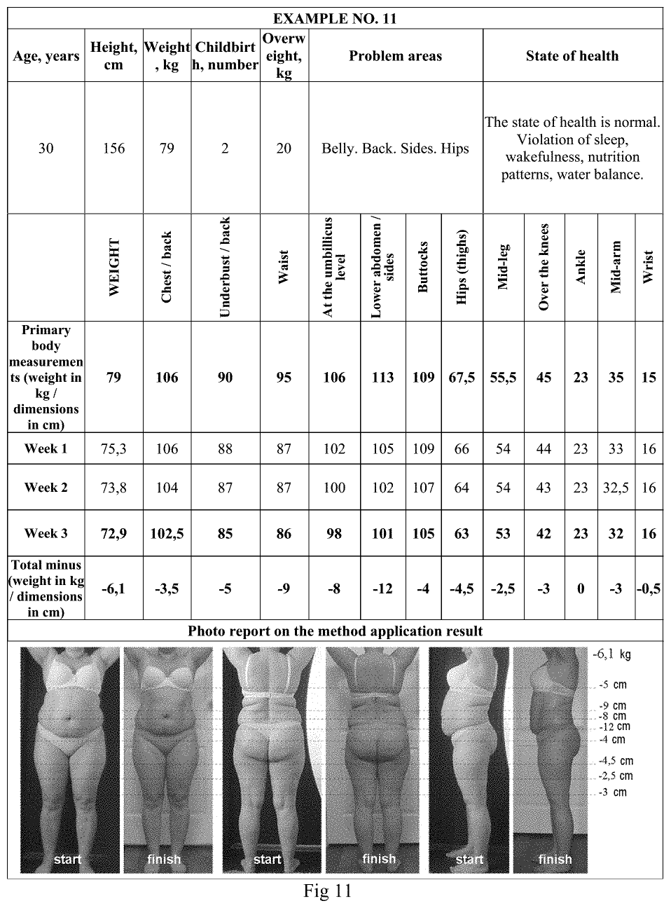

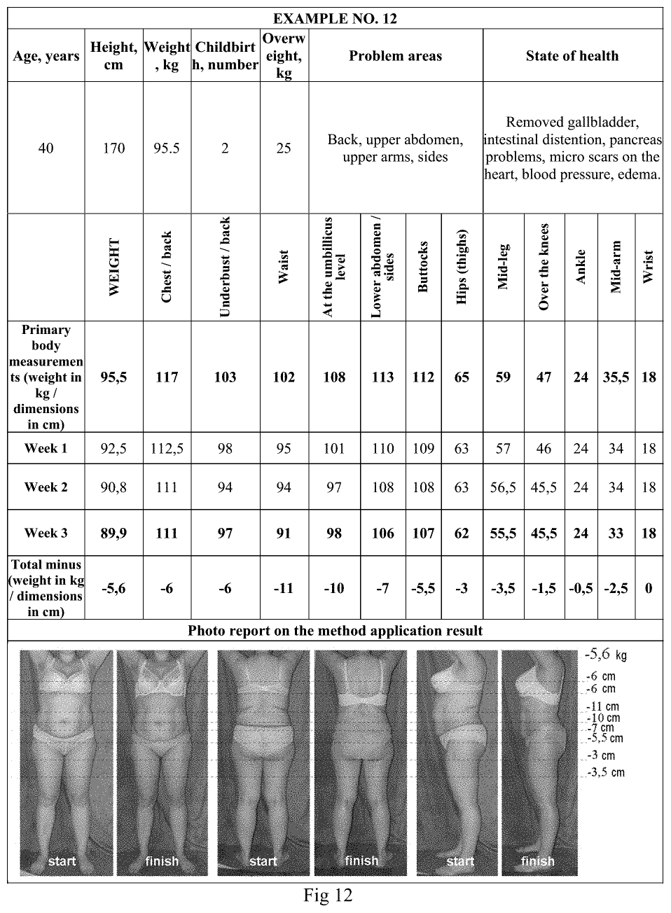

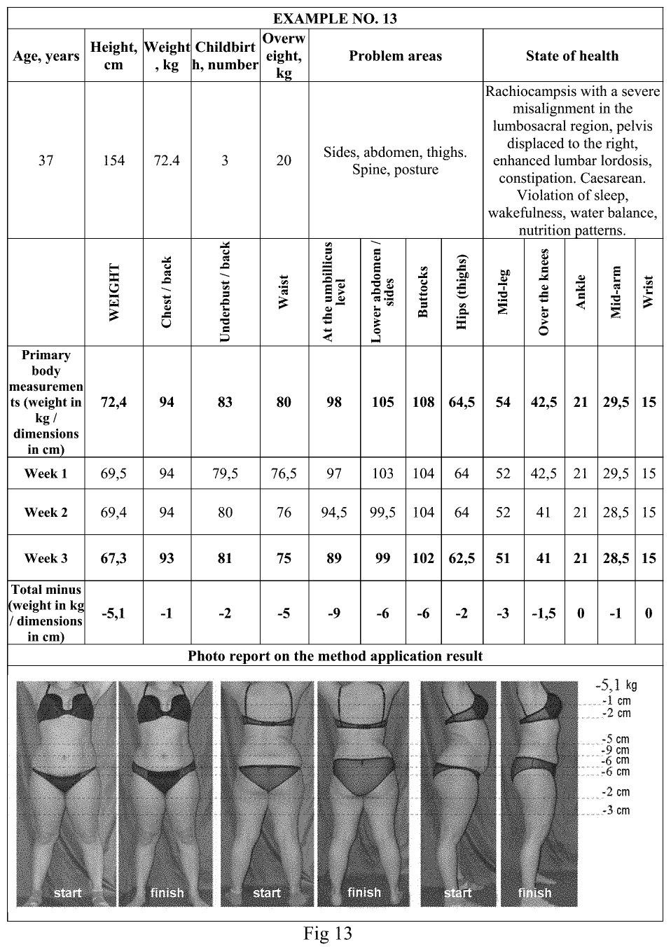

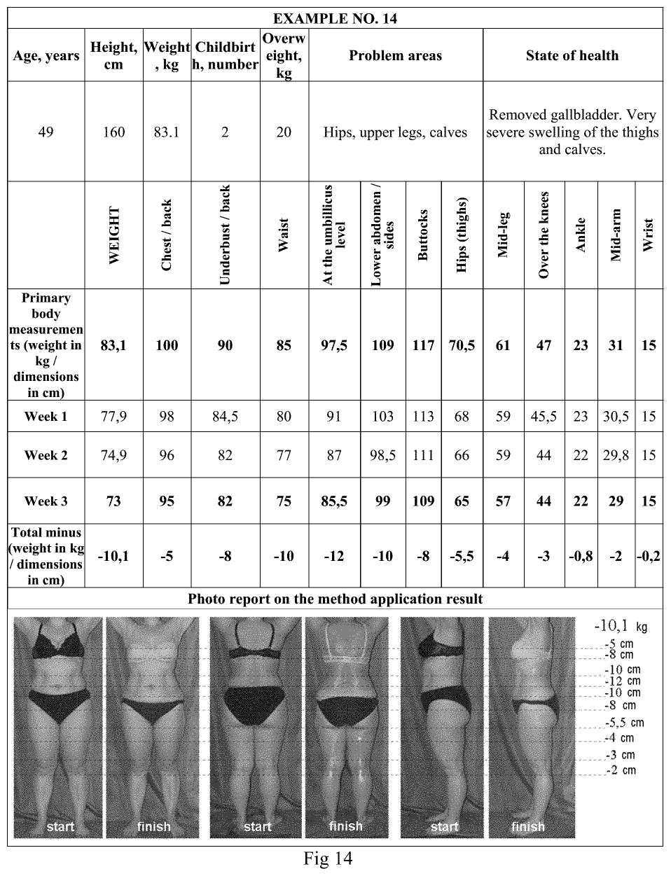

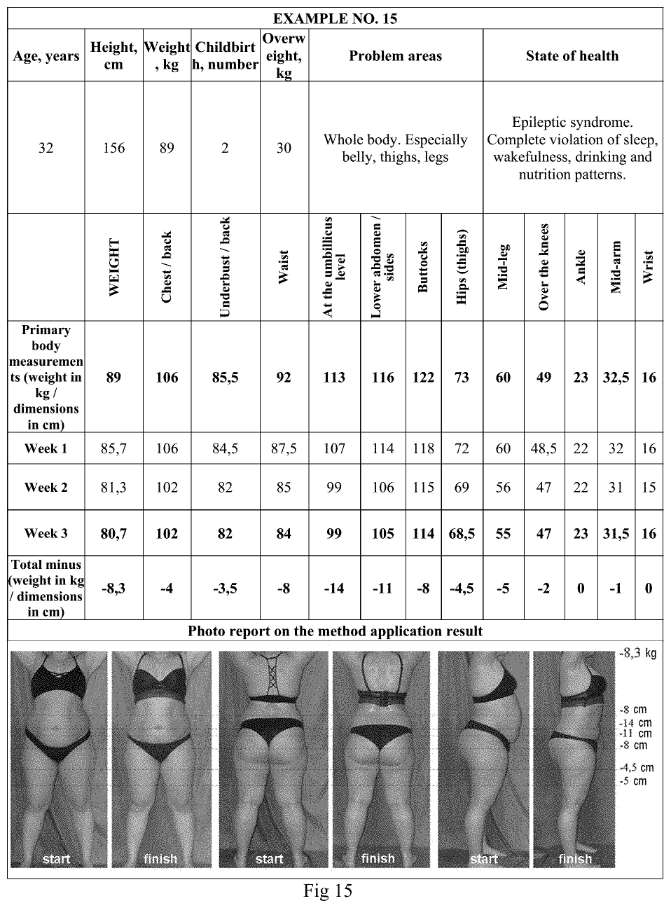

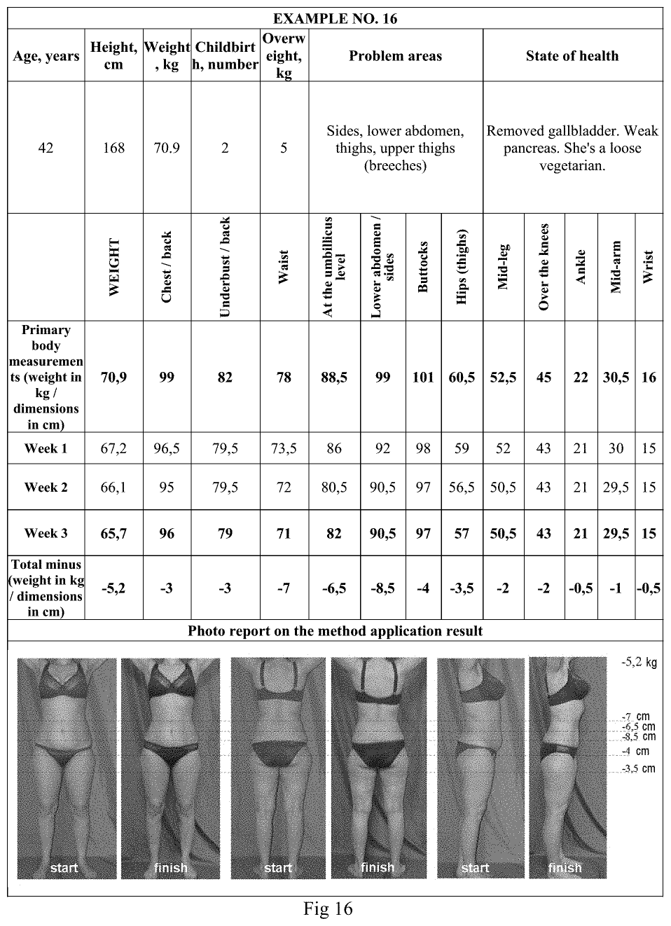

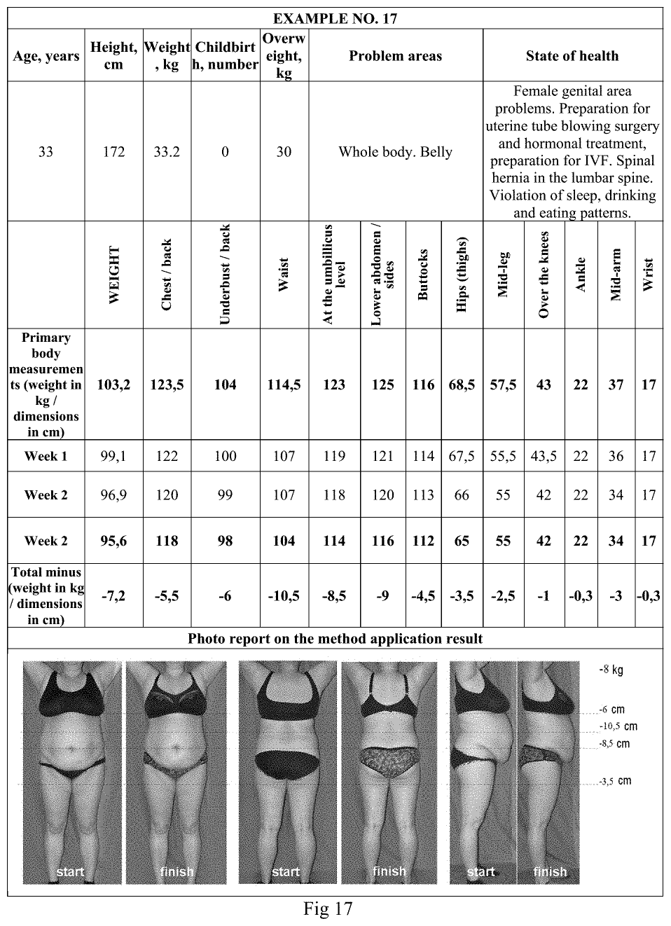

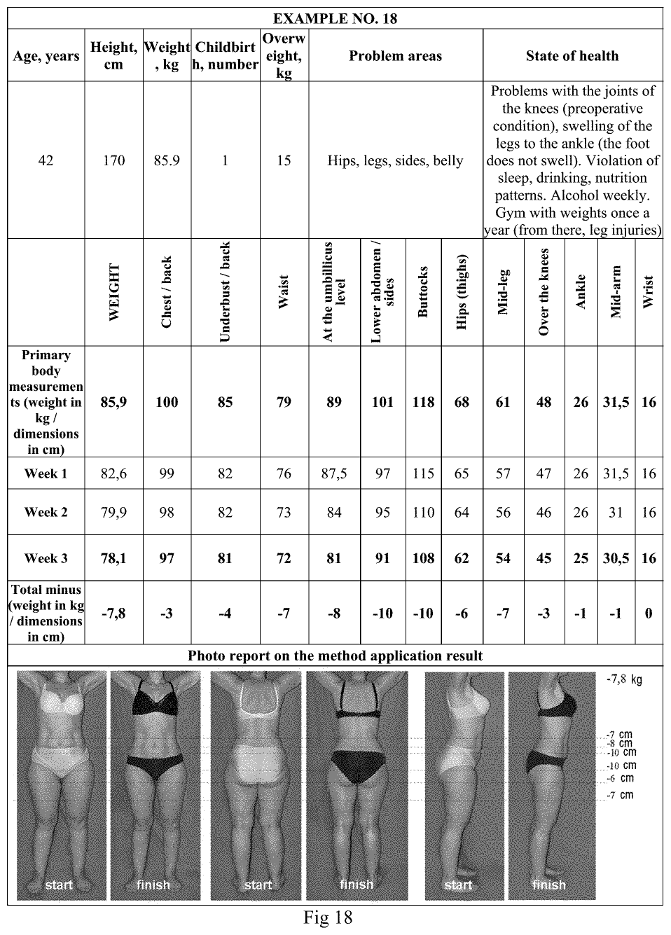

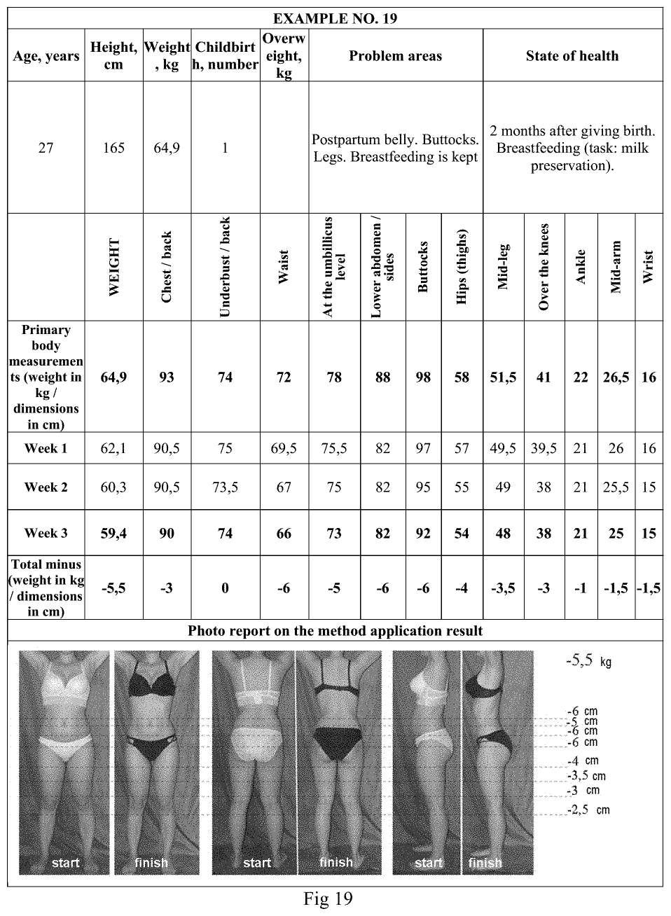

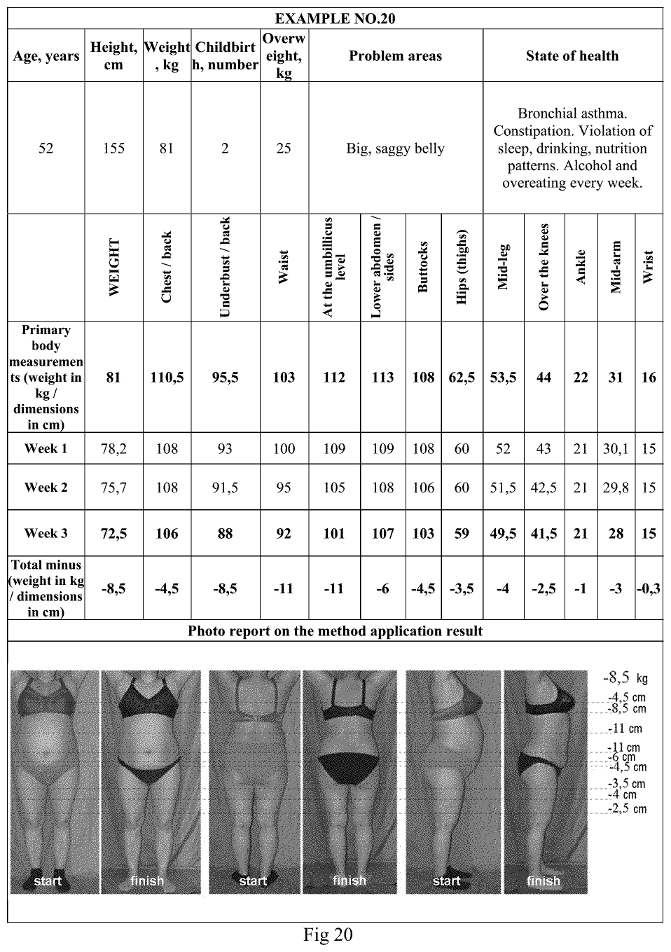

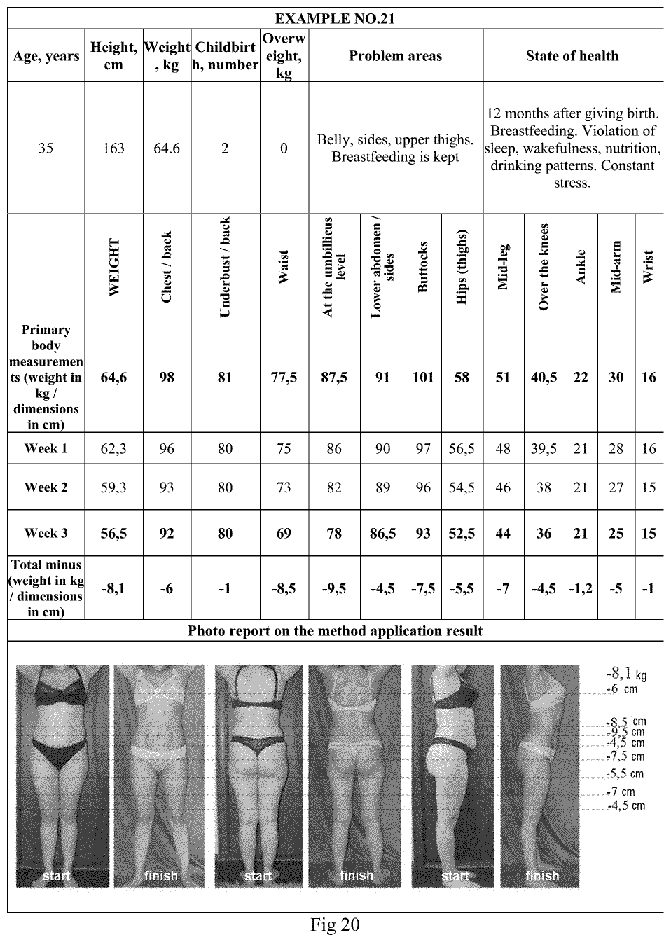

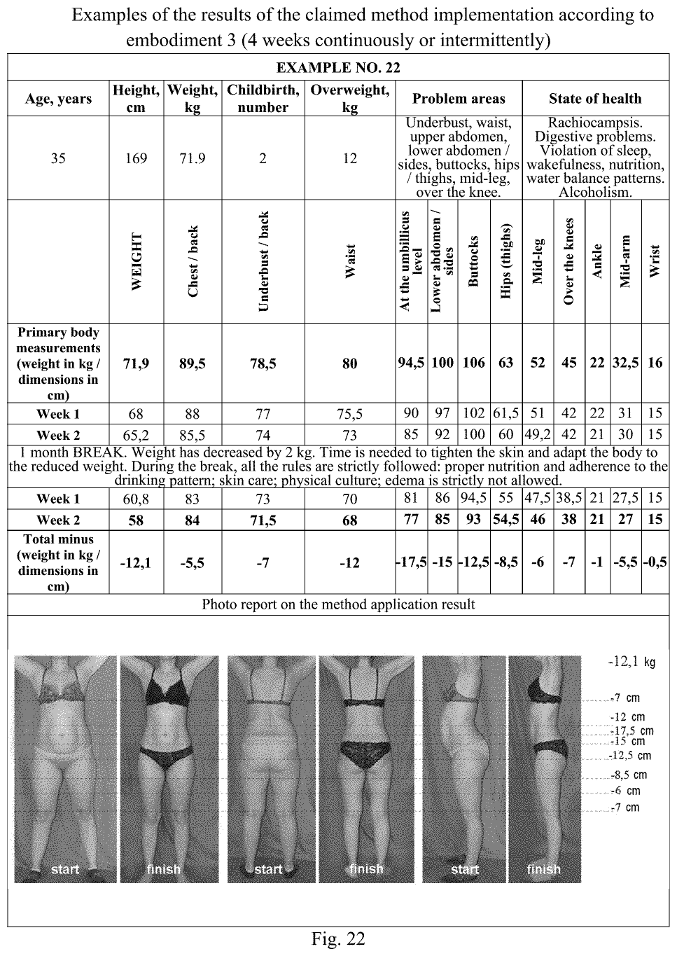

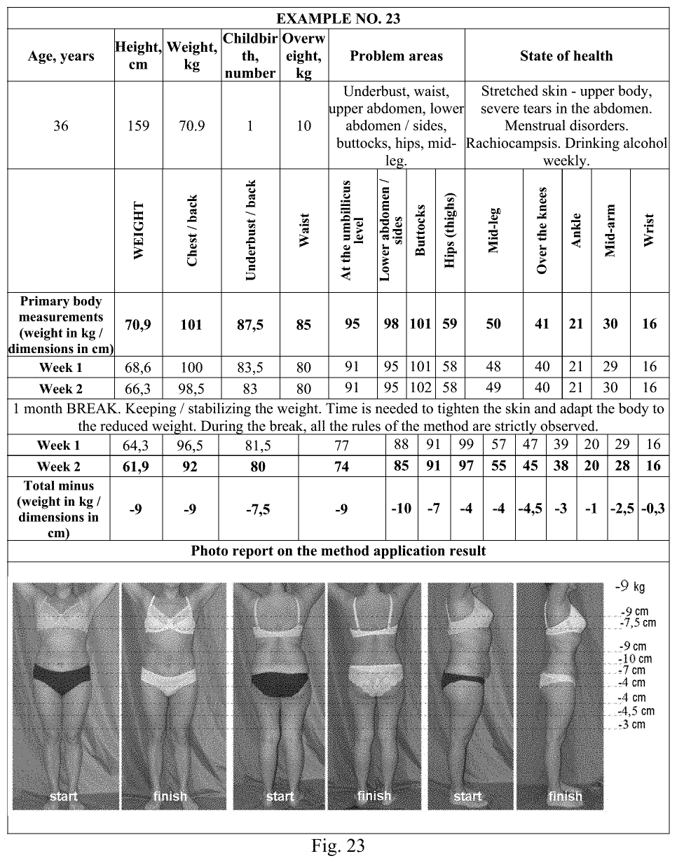

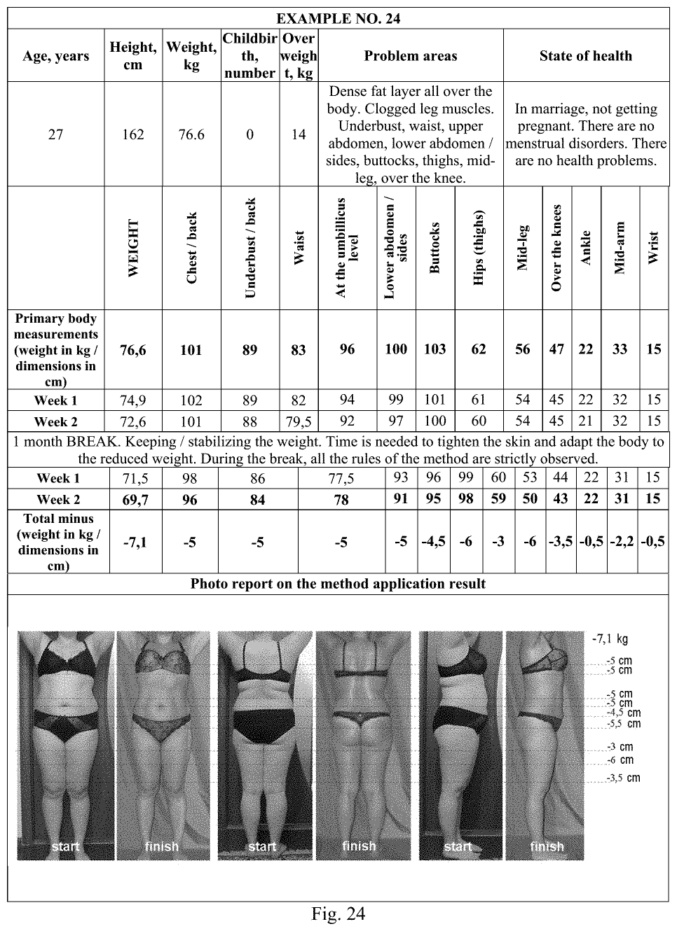

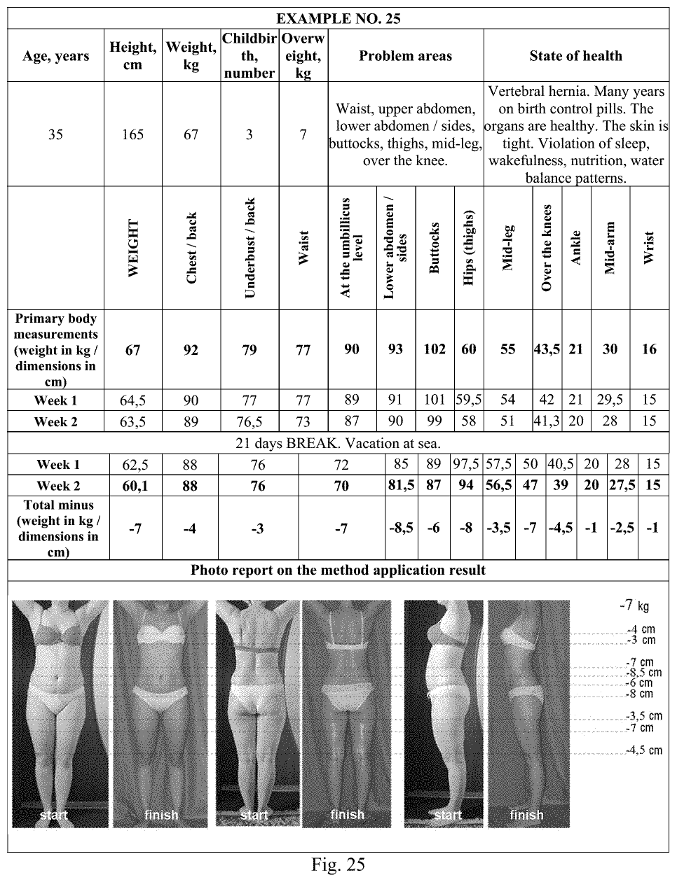

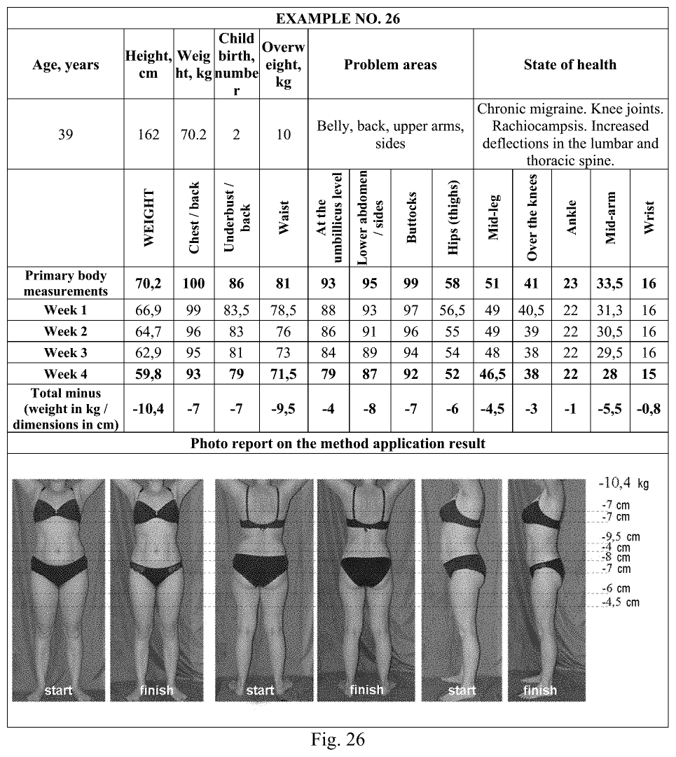

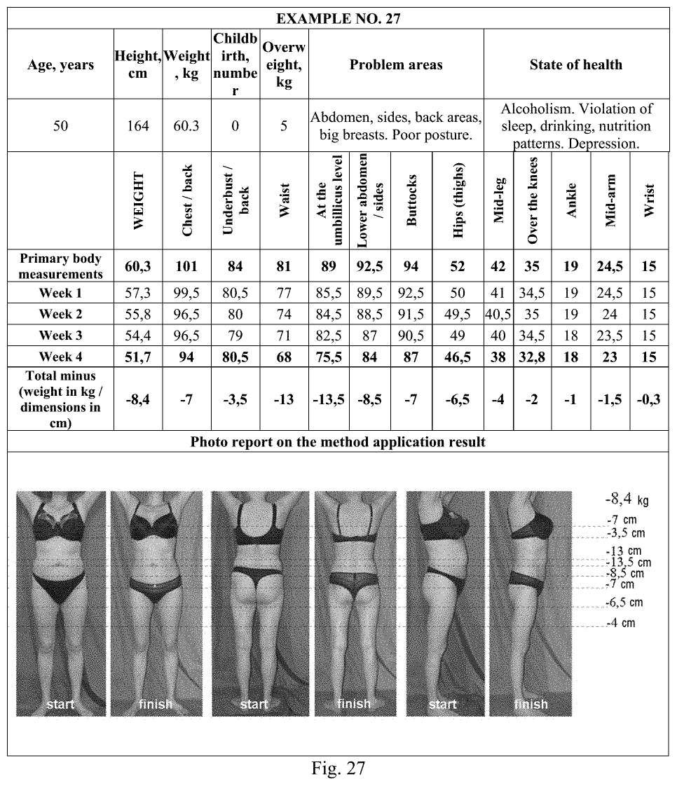

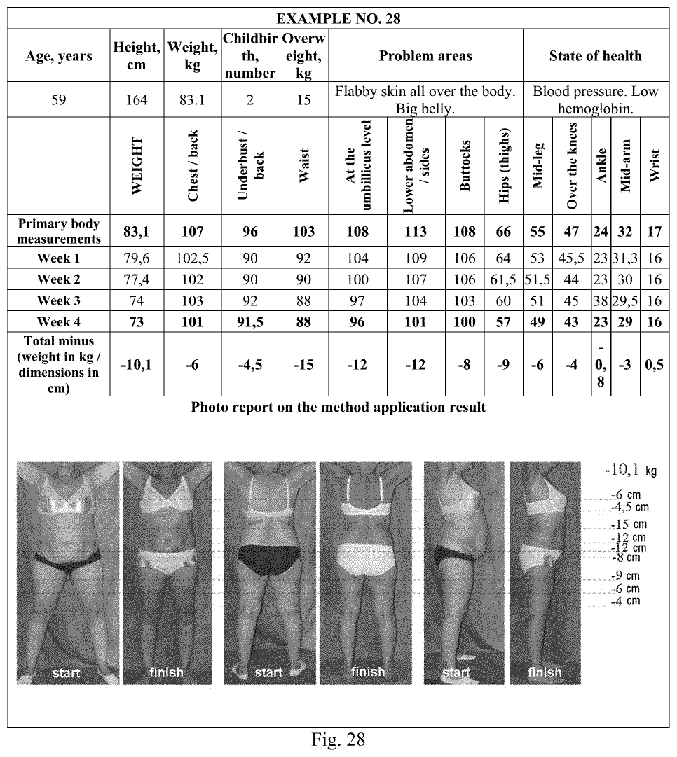

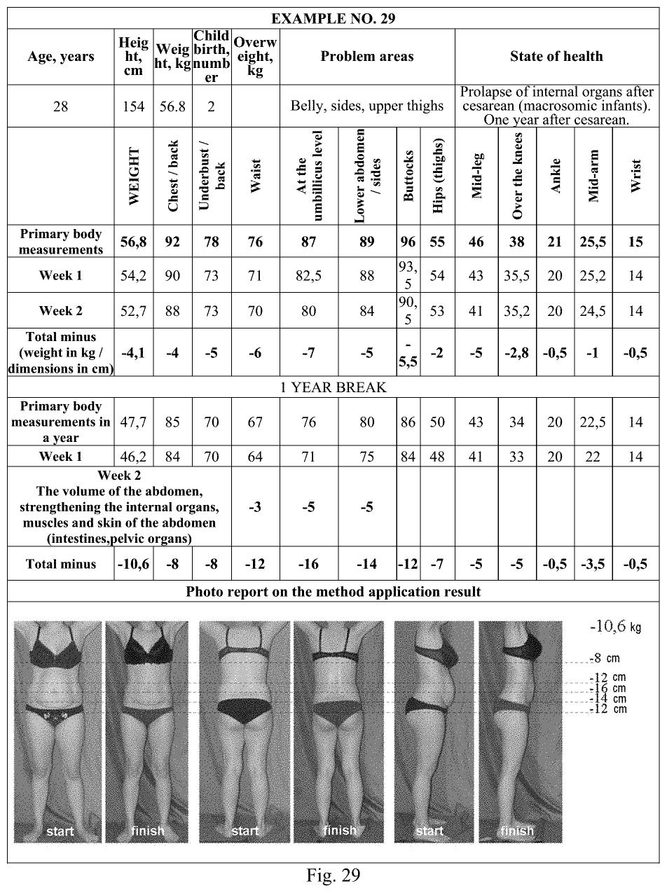

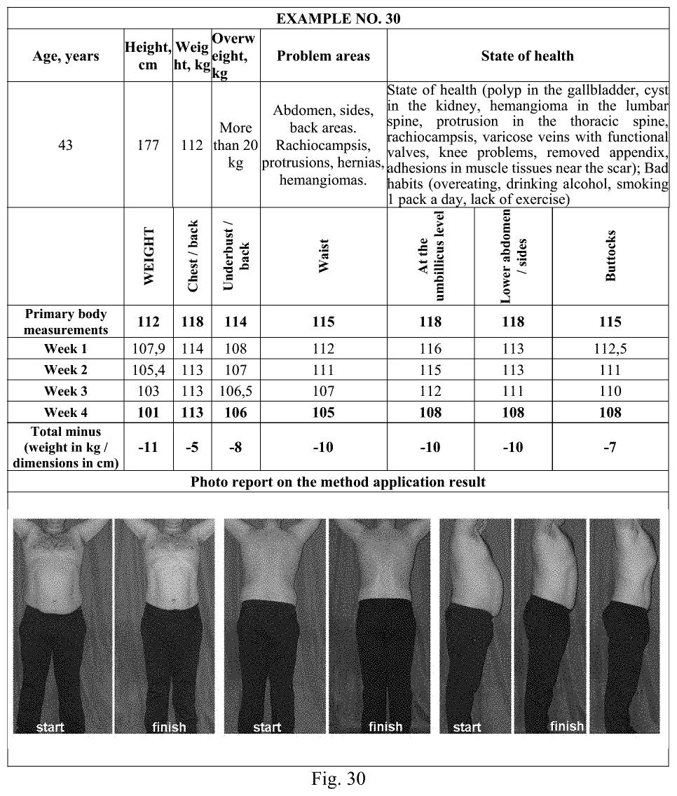

The effectiveness of the claimed method is confirmed using objective data obtained during the implementation of the method, and presented in the control tables in FIGS. 1-30.

### BEST MODE FOR CARRYING OUT THE INVENTION

Depending on the state of health, initial excess weight and body sculpting tasks, the claimed method can be implemented in the form of programs of different duration:

1) embodiment 1: 14 days, recommended in case of overweight up to 5-7 kg, absence of severe chronic diseases and any other diseases in the acute stage

2) embodiment 2: 21 days, recommended in case of 10 kg overweight

3) embodiment 3: 28 days; if necessary, the courses are repeated with a break of 1-3 months; it is recommended in case of overweight from 10-20 kilograms or more, chronic diseases and other health problems.

Therewith, all embodiments provide for daily monitoring and compliance with all patterns (nutrition, water intake, rest, physical activity).

The claimed method also provides for the combination of the above embodiments and/or their alternation, repetition of courses, in particular, in case of a very large overweight.

Below are the tables containing a more detailed presentation of the implementation of the claimed method in specific examples, for each of the embodiments, with examples of recommended measures aimed at body sculpting, weight reduction and overall health improvement of the body, determined based on the results of preliminary analysis and identification of problem areas. The implementation of each of the measures or a set of measures is presented in more detail further in the description.

In all embodiments, on the first day, subjective primary data on the state of health according to the patient and objective data are collected, including initial control measurements: weight, height, dimensions, photos from different angles, as well as informing, instructing and compiling an individual set of measures for body sculpting are provided based on the claimed method.

### Embodiment 1

| | Week 1 | | | | | | | Week 2 | | | | | | |
|---|---|---|---|---|---|---|---|---|---|---|---|---|---|---|
| Stage/Procedure/Exercise | 1 | 2 | 3 | 4 | 5 | 6 | 7 | 1 | 2 | 3 | 4 | 5 | 6 | 7 |
| Collection of information | x | x | x | x | x | x | x | x | x | x | x | x | x | x |
| about the state of health of | | | | | | | | | | | | | | |
| the body, daily updates on | | | | | | | | | | | | | | |
| general condition | | | | | | | | | | | | | | |
| Control measurements | x | | | | | | | x | | | | | | |
| Weighing in the | x | x | x | x | x | x | x | x | x | x | x | x | x | x |
| morning/in the evening | | | | | | | | | | | | | | |
| Vitamins and minerals | | x | x | x | x | x | x | x | x | x | x | x | x | x |
| Use of vegetable oils | | | x | x | x | x | x | x | x | x | x | x | x | x |
| Mono-days | | x | x | x | | | | x | x | x | | | | |
| Balanced nutrition | | | | | x | x | x | | | | x | x | x | x |
| Breathing to regulate heart | x | x | x | x | x | x | x | x | x | x | x | x | x | x |
| rate and blood pressure | | | | | | | | | | | | | | |
| Warming up and starting | x | x | x | x | x | x | x | x | x | x | x | x | x | x |
| intense sweating | | | | | | | | | | | | | | |
| Breathing exercises | | x | x | x | x | x | x | x | x | x | x | x | x | x |
| Massage to improve blood | x | x | x | x | x | x | x | | | | | | | |
| circulation in the muscles | | | | | | | | | | | | | | |
| and to tone the skin | | | | | | | | | | | | | | |
| Massage to enhance | x | x | x | x | x | x | x | | x | | x | | x | |
| lymph flow | | | | | | | | | | | | | | |
| Massage to enhance the | | | | x | x | x | x | x | x | x | x | x | x | x |
| breakdown of the fat layer | | | | | | | | | | | | | | |
| Circular massage of | | | | x | | x | | x | | x | | x | x | x |
| problem areas | | | | | | | | | | | | | | |
| Combining physical | | | x | | x | | x | | x | x | x | x | x | x |
| activity and massage in the | | | | | | | | | | | | | | |
| abdomen area | | | | | | | | | | | | | | |
| Wrapping or | x | | x | | x | | x | | x | | x | | x | |
| instrumentation techniques | | | | | | | | | | | | | | |
| for problem areas | | | | | | | | | | | | | | |
| Skin care, contrast | x | x | x | x | x | x | x | x | x | x | x | x | x | x |
| shower, creams | | | | | | | | | | | | | | |
| Exercises to activate | x | x | x | x | x | x | x | x | x | x | x | x | x | x |
| lymph flow | | | | | | | | | | | | | | |
| Spine exercises | x | x | x | x | x | x | x | x | x | x | x | x | x | x |
| Exercises to strengthen the | | | | | x | x | x | x | x | x | x | x | x | x |
| muscles in the back area | | | | | | | | | | | | | | |
| Exercises for the | | | | | | | | | x | x | x | x | x | x |
| abdominal muscles | | | | | | | | | | | | | | |
| Exercises for the internal | | x | x | x | x | x | x | x | x | x | x | x | x | x |
| organs of the | | | | | | | | | | | | | | |
| gastrointestinal tract | | | | | | | | | | | | | | |
| Exercises for legs and hips | x | x | x | x | x | x | x | x | x | x | x | x | x | x |
| that activate lymph flow, | | | | | | | | | | | | | | |
| prevention of venous | | | | | | | | | | | | | | |
| varices | | | | | | | | | | | | | | |
| Exercises to correct | | | | x | x | x | x | x | x | x | x | x | x | x |
| problem areas (legs and | | | | | | | | | | | | | | |
| hips) | | | | | | | | | | | | | | |

### Embodiment 2

| | Week 1 | | | | | | | Week 2 | | | | | | |
|---|---|---|---|---|---|---|---|---|---|---|---|---|---|---|
| Stage/Procedure/Exercise | 1 | 2 | 3 | 4 | 5 | 6 | 7 | 1 | 2 | 3 | 4 | 5 | 6 | 7 |
| Collection of information | x | x | x | x | x | x | x | x | x | x | x | x | x | x |
| about the state of health of | | | | | | | | | | | | | | |
| the body, daily updates on | | | | | | | | | | | | | | |
| general condition | | | | | | | | | | | | | | |
| Control measurements | x | | | | | | | x | | | | | | |
| Weighing in the | x | x | x | x | x | x | x | x | x | x | x | x | x | x |
| morning/in the evening | | | | | | | | | | | | | | |
| Vitamins and minerals | | x | x | x | x | x | x | x | x | x | x | x | x | x |
| Use of vegetable oils | | | x | x | x | x | x | x | x | x | x | x | x | x |
| Mono-days | | x | x | x | | | | x | x | x | x | | | |
| Balanced nutrition | | | | | x | x | x | | | | | x | x | x |
| Cleansing of the | | | | | | | | | x | x | | | | |
| gastrointestinal tract | | | | | | | | | | | | | | |
| organs | | | | | | | | | | | | | | |
| Breathing to regulate heart | x | x | x | x | x | x | x | x | x | x | x | x | x | x |
| rate and blood pressure | | | | | | | | | | | | | | |
| Warming up and starting | x | x | x | x | x | x | x | x | x | x | x | x | x | x |
| intense sweating | | | | | | | | | | | | | | |
| Breathing exercises | | x | x | x | x | x | x | x | x | x | x | x | x | x |
| Massage to improve blood | x | x | x | x | x | x | x | | x | | x | | x | x |
| circulation in the muscles | | | | | | | | | | | | | | |
| and to tone the skin | | | | | | | | | | | | | | |
| Massage to enhance | x | x | x | x | x | x | x | x | x | x | x | x | x | x |
| lymph flow | | | | | | | | | | | | | | |
| Massage to enhance the | | | | x | x | x | x | | x | | x | | x | x |
| breakdown of the fat layer | | | | | | | | | | | | | | |
| Circular massage of | | | | x | | x | | x | | x | | x | | x |
| problem areas | | | | | | | | | | | | | | |
| Combining physical | | | | | | | x | | x | | x | | x | x |
| activity and massage in the | | | | | | | | | | | | | | |
| abdomen area | | | | | | | | | | | | | | |
| Wrapping or | x | | x | | x | | x | | x | | x | | x | |
| instrumentation techniques | | | | | | | | | | | | | | |
| for problem areas | | | | | | | | | | | | | | |
| Skin care, contrast shower, | | x | x | x | x | x | x | x | x | x | x | x | x | x |
| creams | | | | | | | | | | | | | | |
| Exercises to activate lymph | x | x | x | x | x | x | x | x | x | x | x | x | x | x |
| flow | | | | | | | | | | | | | | |
| Spine exercises | | | x | x | x | x | x | x | x | x | x | x | x | x |
| Exercises to strengthen the | | | | | | | | x | | x | | x | | x |
| muscles in the back area | | | | | | | | | | | | | | |
| Exercises for the | | | | | | | | | | | | | | x |
| abdominal muscles | | | | | | | | | | | | | | |
| Exercises for the internal | | x | x | x | x | x | x | x | x | x | x | x | x | x |
| organs of the | | | | | | | | | | | | | | |
| gastrointestinal tract | | | | | | | | | | | | | | |
| Exercises for legs and hips | x | x | x | x | x | x | x | x | x | x | x | x | x | x |
| that activate lymph flow, | | | | | | | | | | | | | | |
| prevention of venous | | | | | | | | | | | | | | |
| varices | | | | | | | | | | | | | | |
| Exercises to correct | x | x | x | x | x | x | x | x | x | x | x | x | x | x |
| problem areas | | | | | | | | | | | | | | |
| (legs and hips) | | | | | | | | | | | | | | |
| | Week 3 | | | | | | | | | | | | | |
| Stage/Procedure/Exercise | 1 | 2 | 3 | 4 | 5 | 6 | 7 | | | | | | | |
| Collection of information | x | x | x | x | x | x | x | | | | | | | |
| about the state of health of | | | | | | | | | | | | | | |
| the body, daily updates on | | | | | | | | | | | | | | |
| general condition | | | | | | | | | | | | | | |
| Control measurements | x | | | | | | | | | | | | | |
| Weighing in the | x | x | x | x | x | x | x | | | | | | | |
| morning/in the evening | | | | | | | | | | | | | | |
| Vitamins and minerals | x | x | x | x | x | x | x | | | | | | | |
| Use of vegetable oils | x | x | x | x | x | x | x | | | | | | | |
| Mono-days | x | x | x | | | | | | | | | | | |
| Balanced nutrition | | | | x | x | x | x | | | | | | | |
| Cleansing of the | | | | | | | | | | | | | | |
| gastrointestinal tract | | | | | | | | | | | | | | |
| organs | | | | | | | | | | | | | | |
| Breathing to regulate heart | x | x | x | x | x | x | x | | | | | | | |
| rate and blood pressure | | | | | | | | | | | | | | |
| Warming up and starting | x | x | x | x | x | x | x | | | | | | | |
| intense sweating | | | | | | | | | | | | | | |
| Breathing exercises | x | x | x | x | x | x | x | | | | | | | |
| Massage to improve blood | | x | | x | | x | | | | | | | | |
| circulation in the muscles | | | | | | | | | | | | | | |
| and to tone the skin | | | | | | | | | | | | | | |
| Massage to enhance lymph | | x | | x | | | | | | | | | | |
| flow | | | | | | | | | | | | | | |
| Massage to enhance the | | x | x | x | x | x | x | | | | | | | |
| breakdown of the fat layer | | | | | | | | | | | | | | |
| Circular massage of | | x | | x | | x | | | | | | | | |
| problem areas | | | | | | | | | | | | | | |
| Combining physical | x | x | x | x | x | x | x | | | | | | | |
| activity and massage in the | | | | | | | | | | | | | | |
| abdomen area | | | | | | | | | | | | | | |
| Wrapping or | x | | x | | x | | | | | | | | | |
| instrumentation techniques | | | | | | | | | | | | | | |
| for problem areas | | | | | | | | | | | | | | |
| Skin care, contrast shower, | x | x | x | x | x | x | x | | | | | | | |
| creams | | | | | | | | | | | | | | |
| Exercises to activate lymph | x | x | x | x | x | x | x | | | | | | | |
| flow | | | | | | | | | | | | | | |
| Spine exercises | | | x | x | x | x | x | | | | | | | |
| Exercises to strengthen the | | x | | x | | x | x | | | | | | | |
| muscles in the back area | | | | | | | | | | | | | | |
| Exercises for the | | x | | x | | x | x | | | | | | | |
| abdominal muscles | | | | | | | | | | | | | | |
| Exercises for the internal | x | x | x | x | x | x | x | | | | | | | |
| organs of the | | | | | | | | | | | | | | |
| gastrointestinal tract | | | | | | | | | | | | | | |
| Exercises for legs and hips | x | x | x | x | x | x | x | | | | | | | |
| that activate lymph flow, | | | | | | | | | | | | | | |
| prevention of venous | | | | | | | | | | | | | | |
| varices | | | | | | | | | | | | | | |
| Exercises to correct | x | x | x | x | x | x | x | | | | | | | |
| problem areas (legs and | | | | | | | | | | | | | | |
| hips) | | | | | | | | | | | | | | |

### Embodiment 3

| | Week 1 | | | | | | | Week 2 | | | | | | |
|---|---|---|---|---|---|---|---|---|---|---|---|---|---|---|
| Stage/Procedure/Exercise | 1 | 2 | 3 | 4 | 5 | 6 | 7 | 1 | 2 | 3 | 4 | 5 | 6 | 7 |
| Collection of information | x | x | x | x | x | x | x | x | x | x | x | x | x | x |
| about the state of health of | | | | | | | | | | | | | | |
| the body, daily updates on | | | | | | | | | | | | | | |
| general condition | | | | | | | | | | | | | | |
| Control measurements | | x | | | | | | x | | | | | | |
| Weighing in the | | x | x | x | x | x | x | x | x | x | x | x | x | x |
| morning/in the evening | | | | | | | | | | | | | | |
| Vitamins and minerals | | x | x | x | x | x | x | x | x | x | x | x | x | x |
| Use of vegetable | | x | x | x | x | x | x | x | x | x | x | x | x | x |
| oils/nutritional | | | | | | | | | | | | | | |
| supplements | | | | | | | | | | | | | | |
| Mono-days | | x | x | x | | | | x | x | x | | | | |
| Balanced nutrition | | | | | x | x | x | | | | x | x | x | x |
| Cleansing of the | | | | | | | | | x | | | | | |
| gastrointestinal tract | | | | | | | | | | | | | | |
| organs | | | | | | | | | | | | | | |
| Breathing to regulate heart | | x | x | x | x | x | x | x | x | x | x | x | x | x |
| rate and blood pressure | | | | | | | | | | | | | | |
| Warming up and starting | | x | x | x | x | x | x | x | x | x | x | x | x | x |
| intense sweating | | | | | | | | | | | | | | |
| Breathing exercises | | x | x | x | x | x | x | x | x | x | x | x | x | x |
| Massage to improve blood | | x | | x | | x | x | | x | | x | | x | x |
| circulation in the muscles | | | | | | | | | | | | | | |
| and to tone the skin | | | | | | | | | | | | | | |
| Massage to enhance lymph | | x | | x | | x | x | | x | | x | | x | x |
| flow | | | | | | | | | | | | | | |
| Massage to enhance the | | | | x | | x | x | | x | | x | | x | x |
| breakdown of the fat layer | | | | | | | | | | | | | | |
| Massage of the back | | | | | | | x | | | | | | | x |
| muscles and | | | | | | | | | | | | | | |
| gastrointestinal tract | | | | | | | | | | | | | | |
| organs | | | | | | | | | | | | | | |
| Combining physical | | | | | | | x | | x | | x | | x | x |
| activity and massage in the | | | | | | | | | | | | | | |
| abdomen area | | | | | | | | | | | | | | |
| Wrapping or | | x | | x | | x | | | x | | x | | x | |
| instrumentation techniques | | | | | | | | | | | | | | |
| for problem areas | | | | | | | | | | | | | | |
| Skin care, contrast shower, | | | x | x | x | x | x | x | x | x | x | x | x | x |
| creams | | | | | | | | | | | | | | |
| Exercises to activate lymph | x | x | x | x | x | x | x | x | x | x | x | x | x | x |
| flow | | | | | | | | | | | | | | |
| Spine exercises | | | | x | | x | x | | x | | x | | x | x |
| Exercises to strengthen the | | | | x | x | x | x | x | x | x | x | x | x | x |
| muscles in the back area | | | | | | | | | | | | | | |
| Exercises for the | | | | | | | | | | | | | | |
| abdominal muscles | | | | | | | | | | | | | | |
| Exercises for the internal | | x | x | x | x | x | x | x | x | x | x | x | x | x |
| organs of the | | | | | | | | | | | | | | |
| gastrointestinal tract | | | | | | | | | | | | | | |
| Exercises for legs and hips | | x | x | x | x | x | x | x | x | x | x | x | x | x |
| | Week 3 | | | | | | | Week 4 | | | | | | |
| Stage/Procedure/Exercise | 1 | 2 | 3 | 4 | 5 | 6 | 7 | 1 | 2 | 3 | 4 | 5 | 6 | 7 |
| Collection of information | x | x | x | x | x | x | x | x | x | x | x | x | x | x |
| about the state of health of | | | | | | | | | | | | | | |
| the body, daily updates on | | | | | | | | | | | | | | |
| general condition | | | | | | | | | | | | | | |
| Control measurements | x | | | | | | | x | | | | | | x |
| Weighing in the | x | x | x | x | x | x | x | x | x | x | x | x | x | x |
| morning/in the evening | | | | | | | | | | | | | | |
| Vitamins and minerals | x | x | x | x | x | x | x | x | x | x | x | x | x | x |
| Use of vegetable oils | x | x | x | x | x | x | x | x | x | x | x | x | x | x |
| Mono-days | x | x | x | | | | | | | | | | | |
| Balanced nutrition | | | | x | x | x | x | x | x | x | x | x | x | x |
| Cleansing of the | | | | | | | | | | | | | | |
| gastrointestinal tract | | | | | | | | | | | | | | |
| organs | | | | | | | | | | | | | | |
| Breathing to regulate heart | x | x | x | x | x | x | x | | x | | x | | x | |
| rate and blood pressure | | | | | | | | | | | | | | |
| Warming up and starting | x | x | x | x | x | x | x | x | x | x | x | x | x | x |
| intense sweating | | | | | | | | | | | | | | |
| Breathing exercises | x | x | x | x | x | x | x | x | | x | | x | | |
| Massage to improve blood | | x | | x | | x | x | | x | | x | | x | x |
| circulation in the muscles | | | | | | | | | | | | | | |
| and to tone the skin | | | | | | | | | | | | | | |
| Massage to enhance lymph | | | | | | | | | | | | | | |
| flow | | | | | | | | | | | | | | |
| Massage to enhance the | | x | | x | | x | x | | x | | x | | | |
| breakdown of the fat layer | | | | | | | | | | | | | | |
| Massage of the back | | | | | | | x | | x | | x | | | x |
| muscles and | | | | | | | | | | | | | | |
| gastrointestinal tract | | | | | | | | | | | | | | |
| organs | | | | | | | | | | | | | | |
| Combining physical | | x | | x | | x | x | | x | | x | | x | x |
| activity and massage in the | | | | | | | | | | | | | | |
| abdomen area | | | | | | | | | | | | | | |
| Wrapping or | | x | | x | | x | | | x | | x | | x | |
| instrumentation techniques | | | | | | | | | | | | | | |
| for problem areas | | | | | | | | | | | | | | |
| Skin care, contrast shower, | x | x | x | x | x | x | x | x | x | x | x | x | x | x |
| creams | | | | | | | | | | | | | | |
| Exercises to activate lymph | | | | | | | | | | | | | | |
| flow | | | | | | | | | | | | | | |
| Spine exercises | | x | | x | | x | x | | x | | x | | x | x |
| Exercises to strengthen the | x | x | x | x | x | x | x | x | x | x | x | x | x | x |
| muscles in the back area | | | | | | | | | | | | | | |
| Exercises for the | | x | | x | | x | x | x | x | x | x | x | x | x |
| abdominal muscles | | | | | | | | | | | | | | |
| Exercises for the internal | x | x | x | x | x | x | x | x | x | x | x | x | x | x |
| organs of the | | | | | | | | | | | | | | |
| gastrointestinal tract | | | | | | | | | | | | | | |
| Exercises for legs and hips | x | | x | | x | | | x | | x | | x | | |

It should be noted that the measures presented in the tables, when implementing the claimed method, show specific examples, that is, particular cases of implementation, which may vary depending on the individual characteristics and the initial primary data of the client, for whom a set of measures for body sculpting is made. In particular, tactile impact techniques and physical exercises are selected based on the existing problem areas that require correction.

To cleanse the excretory systems of the body, various procedures are carried out, as a result of which the functioning of all organs is improved, the metabolism and the process of weight reduction are faster, more efficient and with a longer retention of results.

Such procedures and actions include the rejection of the use of antiperspirants, the stimulation of intense sweating during physical exercises, a daily shower, body scrubs, a bath and a sauna, while observing a temperature range not exceeding 60 C⁰.

In addition, it is important to improve the function of the urinary system and intestines. In case of chronic constipation, at the beginning of the application of the method, it is necessary to cleanse the large intestine, and then the small one. To do this, one can use an enema and cleanse, for example, according to the Walker method (https://www.ayzdorov.ru/ochishenie_kishechnik_klizmoi.php). To those who suffer from long constipation, it is better to give 3-5 micro-enemas with vegetable oil using a bulb syringe in order to free the rectum from old, already dehydrated feces.

As noted earlier, the fundamental principles of nutrition are based on reducing the portion size to 150-200 ml (200-250 ml for men) and increasing the number of meals up to 5-6 times a day. “Heavy” foods, whose digestion time in the stomach is more than two hours, are excluded from the diet. The diet includes complex carbohydrates that contain fiber, pectins and glycogen: vegetables and herbs, preferably without heat treatment, including in the form of green smoothies, root vegetables—in moderation, legumes and grains (cereals: oatmeal, buckwheat, bulgur, wheat, barley porridge, diet bread/crispbread and pasta made from wholemeal flour/durum wheat; brown rice; beans, peas, snap beans, lentils, chickpeas), dairy products (low-fat cottage cheese, kefir, white yoghurts, unleavened Adyghe cheese), fresh fruits and berries, sea fish (white and red) and/or lean beef meat, liver, vegetable fats in moderation (pumpkin seed oil, olive oil, flaxseed oil, cold-pressed coconut oil, mustard oil, sea buckthorn oil, cedar oil, walnut oil). For persons with intestinal problems, it is recommended to completely eliminate meat. Protein can be obtained from fish, cottage cheese, cheese, legumes.

The recommended diet is as follows:

1. An early breakfast is required at 7-8 am (approximately 30-60 minutes after waking up).

2. Dinnerette is excluded. The last meal (dinner) is taken no later than 19-00.

3. Meals are not washed down! 10-15 minutes before eating, it is advisable to drink warm clear water (37 C⁰). But after eating, it is not allowed to drink either water or other fluids. It is important that the food eaten is fully digested. Any liquid that comes immediately after a meal stretches the stomach capacity, dilutes gastric juices, and impairs digestion.

4. Food must be thoroughly chewed.

5. Balanced, varied diet. Regular meals.

6. It is necessary to completely exclude from consumption trans fats, salt, sugar; canned food, flour-based (except for diet bread/crispbreads and pasta made from wholemeal flour/durum wheat 1-3 times a week before 12 noon), starchy foods (except for carrots and beets if intestinal motility is impaired), fast food, alcohol. It is advisable to reduce the consumption of foods containing gluten, up to a complete elimination of them for those suffering from gluten intolerance.

Example of a daily diet.

1. Early breakfast is required, 7-8 am. Complex carbohydrates are preferred. Breakfast options: porridge or an omelet with green vegetables, or durum wheat pasta. Breakfast should be satisfying enough, but not heavy.

2. Morning snack at 9-10 am. Morning snack options include fresh/baked fruits, fruit salads/cheese sandwich, and coffee/tea with dried fruits. Individually, in case of constipation, baked pumpkin, apples, kiwis, plums are recommended.

3. Second snack at 11-12 am. Second snack options: fruits/cheese sandwich and coffee/tea with dried fruits.

4. Lunch at 1-2 pm. Fats and proteins in a high-quality combination with fiber: fish, dietary meat (chicken, veal, rabbit, liver, chicken liver) baked or steamed with green vegetables; vegetable soups (optionally with meat); casseroles.

5. Third snack at 3-14 pm. Preferably vegetables, light/vegetable proteins (low-fat cottage cheese/legumes, lentils), dried nuts in moderation. Fruits for consumption: baked apples, grapefruits, plums.

6. Dinner at 6-7 pm. Low-fat fermented milk products that improve digestion (thermostatic, white, bio-yoghurts; fresh kefir 1.5-2%). Individually, in cases of constipation, vegetables that stimulate the intestines are recommended, such as boiled beets with the addition of a small amount of olive oil; stewed vegetable mixes, seasoned with white yogurt or a little olive oil. In case of intestinal diseases (constipation, hemorrhoids, colitis), proteins of animal origin are excluded in the evening.

If the recommendations are followed, the intake of fats and protein is significantly reduced in the diet. Protein and fats are present, but they come in the body in a more sparing form and in moderation. To reduce the load on the kidneys during active fat breakdown and to cleanse the body of waste products, salt is removed from the diet (especially for those who have problems with the urinary system, suffering from edema and lymph congestion). Simple, unhealthy carbohydrates are completely excluded. The emphasis is on dishes in which the minimum amount of ingredients is mixed, which reduces the likelihood of mixing incompatible products and helps the body to digest food more easily, assimilate everything necessary and useful that enters the body without unnecessarily overloading it.

Since the claimed method greatly reduces the amount of fats taken, the lack of fat in a balanced diet is compensated for by higher quality, healthy and easily digestible fats. Persons who have problems with the biliary tract or with the gallbladder removed are advised to consume vegetable oils no more than 1-2 times a day with food, very sparingly. Vegetable and aroma oils are also used in large quantities in skin care and tactile techniques.

The most useful and effective oils for use in the process of implementing the claimed method and recommendations for their use are as follows:

Pumpkin seed oil: 0.5-1 teaspoon is taken on an empty stomach in the morning after taking warm clear water every day. With the gallbladder removed or acute or chronic cholecystitis, pumpkin seed oil should not be taken. The oil contains a large amount of biologically active substances: phospholipids, vitamins B1, B2, C, P, flavonoids, unsaturated and polyunsaturated fatty acids: linolenic, oleic, linoleic, palmetic, stearic. It has a recognizable smell, the consistency is slightly denser than that of usual vegetable oils.

Flaxseed oil: 0.5-1 teaspoon is taken on an empty stomach in the morning after taking warm clear water every day or every other day, as well as for dressing salads without heat treatment. It contains a large amount of Omega-3 polyunsaturated fatty acids, vitamins E, F, A, B. In case of female genital problems, flaxseed oil should not be taken.

Olive oil: taken in the morning with salads every day. In the evening, it is added to salads from beets, prunes (in cases of constipation).

Mustard oil: Used as a salad dressing. It contains vitamins A, B6, D, K, P.

Sea buckthorn oil: it is recommended to add it to salads in combination with any other vegetable oils, and also to use it for cooking any dishes. It contains carotenoids, vitamins E, F, A, K, D and biologically active substances.

Cedar oil: it is recommended to add it to salads for flavoring, as well as for massage in a steam bath or sauna. It contains a wide range of substances useful for the human body: polyunsaturated fatty acids, proteins, vitamins A, B, E, D, F, 14 amino acids, 19 different microelements.

Another of the most important stages in the method is mono-days, which are used to help the body to engage in the cleansing process. These days, it is very important to follow all the rules and try not to be overwhelmed, both physically and mentally.

1) The first day—oatmeal. Oat flakes “Hercules”, dried apricots, prunes, water. Eating 5-6 times a day, every 1.5-2 hours.

2) The second day—vegetables (stewed/grill/puree soup). Vegetables, aromatic herbs, allspice, garlic, greenstuff. Eating 5-6 times a day, every 1.5-2 hours.

3) The third day—fresh vegetables, greenstuff. Eating 6-7 times a day, every 1-1.5 hours.

It is necessary to avoid sugar, salt, oil, mayonnaise, starchy condiments, thickeners or salt. If the method is implemented in the form of a course, then the use of mono-days is repeated every week, but no more than 3 times. During this period, the structure of fat in the body changes noticeably due to a sharp decrease in calorie content; the system for removing congestions is activated, since the gastrointestinal tract is not overloaded and functions more efficiently; the metabolism starts to gradually speed up; superficial edema decreases, because products that retain fluid in the body are excluded. When carrying out mono-days, it is obligatory to take clear water and a vitamin-mineral complex. It is recommended to drink warm chamomile tea and carminative teas 2-3 times a day as this reduces inflammation and bloating in the intestines.

Furthermore, since the content of nutrients in food products is insufficient to fully obtain the vitamins and minerals that the body needs, it is necessary to additionally take vitamins and food supplements to adjust the intake of the required amount of vitamins and mineral nutrients for the full functioning of all organs.

During the entire period of the implementation of the method, it is very important to consume a sufficient amount of clear (filtered or melting) water. The water should be warm, 35-37 C⁰. Water should only be drunk before eating, on an empty stomach. It is not allowed to drink water late in the evening or at night. It is not advisable to drink water during and immediately after physical activity. It is recommended to drink 1-2 glasses of water 1.5 hours before exercises. This allows to control the blood pressure during exercises. After exercises, it is allowed to rinse the mouth and take no more than a couple of sips. After 30-60 minutes, it is allowed to drink warm water, in small sips: if exercises are conducted in the evening, the volume of fluid to drink is 0.5-1 glasses. If exercises are conducted in the morning or in the afternoon, it is allowed to drink 1-2 glasses of warm water in small sips.

In the claimed method, breathing is one of the main components of the process of improving metabolic processes, recovering, reducing the body weight and sculpting the body. In breathing exercises, a lot of attention is paid to both inspiration and expiration. In exercises with the stress on the corpus muscles, the emphasis is on expiration and breath holding for a few seconds, and at this moment the exercise is performed. This stimulates enhanced metabolic processes, fat burning in the area where the muscles work, and further, during inspiration, the maximum saturation of cells with oxygen. Breathing exercises are performed outdoors, with open windows, on the balcony or outside. Exercises are performed with short breaks, avoiding severe dizziness, so as not to get the opposite effect—hyperventilation of the lungs.

The claimed method includes the use of the following breathing exercises:

Bhastrika Pranayama. Breathing should be through the nose only. The inspiration is arbitrary. The expiration is strong (as if a person wants to blow his/her nose), the ribs are compressed, the abdominal muscles are sharply tense and pull the stomach up. Bhastrika Pranayama is performed using only the lower ribs, lungs, diaphragm and abdomen. One can do it while standing (stretching the arms up, as if stretching the spine, or simply placing them on your hips), or sitting in the lotus position, straightening the spine.

Vacuum in the abdomen. It is performed only on an empty stomach. Having made a support with the hands on half-bent knees, the person needs to exhale strongly all the air through his/her mouth and, holding his/her breath, draw in the stomach as much as possible under the ribs. In this position, while holding the breath, he/she should draw in and throw out the stomach several times, then straighten up, draw a strong breath (through the nose only).

Breathing with the spinal column. Starting position—sitting in the middle of a chair, not pressing against the back, or standing with legs wide apart, feet turned forward. Breathe out and relax. Breathe in and bend the lower part of the sacrum back from the perineum. At the same time, tilt the head back to the shoulders so that the eyes look up, forming an arc in the middle of the back. Move the stomach and chest forward to expand the chest, clench both hands into fists, raise the fists to shoulder level, while bending the elbows, stretch the elbows and shoulders back, as if trying to bring the shoulder blades together, press the neck into the shoulders and clench the teeth. Breathe out and bend the lower part of the sacrum and the head forward, while rounding the back, bring the elbows, forearms and fists forward and bring them together in front of the chest, trying to squeeze the chest as much as possible, and then press the chin into the upper part of the chest. Repeat at least 10 times in one session.

The breathing exercises listed above help the body to burn exactly the visceral fat that envelops the organs, in the event that there is internal obesity. These exercises strengthen the diaphragm and the thin abdominal wall, as well as the corpus and rib muscles.

Body sculpting only by simple weight loss is ineffective, can lead to flabby and sagging skin, and, therefore requires exercise. At the same time, preference is given to general strengthening exercises aimed at strengthening the muscles of the body, joints, ligaments and spine in order to improve the tone and posture and to form a general fit, well-shaped appearance, as well as to correct specific problem areas. Various studies confirm the fact that the heart rate during exercises should not be excessive, as this causes a heavy strain on the body, but reduces the effectiveness of exercises.

Therefore, in the process of weight reduction and body sculpting, it is of great importance to perform physical exercises, aimed at correcting the identified problem areas, after warming up and starting sweating, while keeping the heart rate and blood pressure at average values with the help of breathing. To do this, first, moderate and intense loads are alternated in short phases of 30-60 seconds, and then static strengthening exercises are performed while holding the breath. In the process of performing the first stage with alternating moderate and intense loads, it is imperative to control breathing, taking a deep breath through the nose, filling the chest and abdomen with air, and then breathing out for a long time, 2 times longer than the inspiration, through the mouth, forming a compressed “0” with the lips. Such breathing during training does not dry out the oral mucosa, and also allows to keep the heart rate and blood pressure at average values. As a static exercise, for example, an exercise using a roller is performed, which is repeated from 3 to 9 times: in the initial position, standing on all fours, lean on the roller, arms and legs are perpendicular to the body, knees are apart at shoulder length, then breathe out and hold breath, draw in the stomach and move the roller forward, forming an angle between the body and arms and legs from 100 to 130 degrees, hold for 3 to 12 seconds, keeping the muscles in tension, then return to the initial position and take a deep breath. Good function of the sweat glands of the body allows to effectively control the blood pressure. Intense sweating during moderate strain on the muscles and circulatory system allows to remove more waste products and salts with sweat, thereby reducing the risk of kidney overload. This is very important, since during weight reduction and body sculpting, the liver and kidneys remove most of the toxins. Intensive breathing with access to fresh air makes it possible to remove as much waste products as possible with exhaled carbon dioxide and, at the same time, saturate and supply body tissues with oxygen, which also increases the effectiveness of the measures used for body sculpting.

For general health improvement and body sculpting during the implementation of the claimed method, various stretching exercises, enhancing the lymph flow, joint health exercises, for example, according to the method of Mirkazarim Norbekov, body rubbing exercises, exercises to prevent prolapse of internal organs, improve the functioning of the gastrointestinal tract and prevent hernias of the spine are performed according to the method of Bubnovsky S. M., as well as exercises to strengthen the corpus muscles and sculpt the body, aimed at correcting certain problem areas.

Exercises aimed at correcting certain problem areas include exercises to warm up the side areas (to correct fat deposits in the upper pelvis and sides), exercises to shape the waist area, exercises to shape the neck, chest and shoulders, exercises to shape the leg areas and buttocks.

It should be noted that with a rapid decrease in body volume, there is a risk of sagging skin. In the claimed method, an emphasis is placed on high-quality skin care for the whole body simultaneously on all sides, which makes it possible to achieve the effect of non-surgical skin tightening. Through the proper nutrition, drinking pattern and exercise, the turgor of tissues, muscles and skin increases. The process of cleansing the body tissues, which starts in the very first days of the method, helps to moisturize the body from the inside; toxins are removed, and instead of them pure water, vitamins, minerals enter the tissue cells, which also helps to improve elasticity. Daily normal physical activity, proper nutrition and drinking—all this gradually stabilizes hormones, and many hormones also have an effect on tissue elasticity. Provided that active assistance is given to the body from the inside, it is additionally appropriate to stimulate the skin from the outside in order to obtain the maximum effect. Water procedures, creams, oils, gels for wrapping, algae, etc., recommended in the implementation of the proposed method, stimulate and enhance various qualities of the skin, such as elasticity, firmness, density, which leads to its more active tightening. These products additionally moisturize the skin, which also improves its turgor and protective function. The subcutaneous circulation improves, which stimulates the skin to rejuvenate and tighten; the skin color is evened out. Recommended procedures include, in particular, contrast showers, rubbing with vegetable and aromatic oils, body wraps (for example, bandage, cryo-compression bandage, draining compression bandage, anti-cellulite compression bandage), and wearing compression underwear.

When implementing the claimed method, various tactile techniques are used, both on the body as a whole and on the identified problem areas. As such, massage and instrumentation techniques are used, which help to change the structure of fat, reduce edema and body volume, stimulate skin strengthening, tone it, and improve lymph flow. For example, lymphatic drainage, sports/squeezing, honey, anti-cellulite, sculptural and modeling massages are used, as well as cosmetological equipment for the beauty industry to process fat on certain areas or the whole body. One of the features of the claimed method is that for a more effective impact of various tactile techniques, just before affecting certain problem areas, the muscles in these areas are pre-stimulated to improve blood circulation through physical exercises aimed at modeling the problem area that will be affected. The body is warmed up from the inside due to improved blood circulation and is ready to oxidize fats, as blood circulation and gas exchange in tissues increased during training Muscles in problem areas are stimulated, it is in them that the need for nutrition is most increased. Therefore, when, immediately after the physical warm-up, the problem area is stimulated from the outside, kneading and rubbing the fat layer, making it softer and increasing blood circulation in it, weight reduction and body sculpting are more effective and in those areas where it is required.

If the patient has diastasis of the rectus abdominis muscles (disruption of the inner edges of these muscles), the claimed massage method is used for non-surgical correction, which includes impact on fat deposits, the peritoneum and weakened muscles in the abdomen area in the form of a combination of physical activity and massage. The client lying in the supine position with the straight legs brought together and the hands folded under the back of the head produces a deep, soft expiration from the abdomen, pulls in the abdomen as much as possible. Next, the operator fixes the area of connection of the abdominal muscles, placing a hand in the middle of the client's abdomen: from the pubic bone (elbow), to the stomach area (wrist) and presses the abdomen. The client, holding breath, raises the straightened legs 30 cm from the surface, while raising the head, shoulders, shoulder blades, tensing the abs and the muscles of the ribs, and the operator at this time forcibly presses the client's abdomen with the hand, not allowing the lower back to bend and the abdominal muscles to lift. Then the operator, with the knuckles of the free hand, which is balled into fist, performs a dynamic rubbing in the up and down direction along the abdominal muscles and the white line of the abdomen for a few seconds until the skin reddens, then the same action is performed on the other side. The cycle of this manipulation is repeated several times on both sides.

This massage method allows to quickly reduce the disruption of the rectus abdominis muscles, and the stimulated abdominal muscles do not allow them to immediately disrupt back. Subject to the rules of nutrition, avoiding bloating, constipation and excessive stretching of the stomach, the anterior abdominal wall returns to the aesthetic norm in about 1-3 weeks. From the second week after applying this method, you it is possible to include abdominal exercises in the cycle, consolidating the effect.

With a “big belly”, it is recommended to carry out a consistent cleansing of the internal organs (cleansing the large and small intestines, cleansing the liver). By adjusting the diet, cleansing the internal organs, starting the self-cleansing of the body, active fat burning and weight reduction are achieved, but there is a risk of prolapse of the internal organs. To prevent it, it is necessary to perform exercises to improve the function of the gastrointestinal tract and strengthen the corpus muscles from the very beginning of the application of the method. For this end, basic physiotherapy exercises are used, for example, a prevention complex according to the Bubnovsky method. The following exercise is also effective: lying on the back, rest the feet on a support, for example, a bench/sofa/bed, knees are bent, exhale, lift the pelvis up, draw in the stomach, tense the pelvic floor muscles, squeezing the anus or vagina (for women), then tense the muscles in the umbillicus and hypochondrium, stay in this position for a few seconds (it may be performed while holding breath), lower the pelvis, relax and repeat 10-15 times.

In the process of implementing the method, it is also very important to coordinate all actions with the human biorhythms, monitor compliance with the sleep and rest pattern, and have a sufficient number of hours of night sleep. Physical exercises also have optimal hours, which are recommended to be adhered to: in the morning, from 6-7 am on an empty stomach, lymph and gastrointestinal tract “pumping” is carried out, while performing breathing exercises for 10-15 minutes; stretching for the back (“cobra”/“downward dog”, “salutation to the sun” yoga) for 5-10 minutes, exercises enhancing the lymph flow for 5-15 minutes; at 10-11 am, on an empty stomach, exercises for fat burning are performed, in the afternoon, at 12 am-1 pm, before lunch, on an empty stomach, —exercises for pumping lymph and gastrointestinal tract, in the evening, at 8-10 pm, on an empty stomach, —exercises for fat burning.

An important role in the implementation of the method is played by the collection of information and control. For this end, subjective information is collected according to the client, as well as objective data, including measurements of control parameters: weight, height, dimensions. Weight measurement is carried out without clothes, in underwear, on an empty bladder. It is better to measure weight in the early morning, immediately upon waking up, after emptying the bladder and, if possible, the intestines, and before taking water and/or food. Dimensions are measured at specific points on a weekly basis. Measurement of control parameters allows to track the dynamics of body sculpting. Control of changes in body dimensions makes it possible to timely adjust physical exercises, the intensity of tactile impact on specific areas, to track problems associated with congestion in the lymphatic system, as well as the quality and systematics of all parameters.

Dimensions are measured at the following points: chest/back (at the level of the chest—the most prominent part); underbust/back (chest under the bust, along the bottom edge of the bra); waist; at the level of the umbillicus (dimension at the level of the umbillicus); lower abdomen/sides (5 cm below the umbillicus, the sides from the back-top, the bones of the pelvis and sacrum fall into this dimension); buttocks (along the most prominent points of the buttocks); thigh/top of the leg (retreating 5 cm down from the perineum, the so-called “breeches” and the lymph node of the inner side of the thigh fall into this dimension); mid-leg (mid-thigh); above the knee (above the kneecap); under the knee (under the kneecap, the popliteal lymph node falls into this dimension); ankle (the thinnest part of the leg above the ankle joint); top of the shoulder (5 cm below the bend of the junction of the arm and the clavicle bone); mid-arm (middle of the humerus); wrist (the thinnest part of the hand above the wrist joint).

It is also recommended to control regularly by photographing the body from different angles. This form of control helps to track changes in the figure: posture, decrease in body fat, the state of general appearance. This allows to timely adjust the action plan for body sculpting.

The claimed technical solution is a highly effective comprehensive method for non-surgical impact on the human body in order to reduce weight and sculpt the body, due to the general cleansing of the body from congestions, improving posture, reducing body fat in problem areas and tightening and improving the condition of the skin, which does not require surgical resection after reducing body dimensions. It can be used for body sculpting of both women and men, of any age group and any health condition, even in the presence of chronic diseases.

1. A method for non-surgical body sculpting, in which problem areas of the figure are identified, a set of measures for body sculpting is compiled, including cleansing the excretory systems of the body, regulating water metabolism, diet and rest, unloading the gastrointestinal tract using mono-days in which one product or one type of product is consumed, and improving nutrition, in cases of chronic diseases, the diet prescribed by the attending physician is applied, taking vitamins and completely eliminating alcohol and foods whose digestion time in the stomach is more than 2 hours; eating in portions of 150-200 ml, 5-6 times a day; physical exercises aimed at forming a healthy posture, strengthening the corpus and anterior abdominal wall muscles, as well as working out problem areas; breathing exercises to enhance the burning of visceral fat, improve the supply of oxygen to the body tissues and perform self-massage of internal organs; tactile techniques of impact, while, immediately before carrying out procedures with tactile effects on certain areas, the muscles in these areas are preliminarily stimulated to improve blood circulation by means of physical exercises, before which warm-up is performed and intense sweating is started by alternating moderate and intense loads in phases of 30-60 seconds with heart rate and blood pressure regulation by breathing, followed by static strength exercises during breath holding, wherein water intake during physical exercises is excluded, but is required 1-1.5 hours before them.

2. A method of non-surgical body shaping according to claim 1, characterized in that tactile techniques of impact include, but are not limited to a combined method of tactile stimulation of muscles with simultaneous tension of these muscles, contrast showers, rubbing, wrapping, as well as massage, instrumentation procedures, wearing compression underwear.

3. A massage method for non-surgical body sculpting, which includes the impact on fat deposits, the peritoneum and weakened abdominal muscles by way of a combination of physical activity and massage, in which, in the supine position with the straight legs brought together and the hands folded under the back of the head a deep, soft expiration is produced from the abdomen, the abdomen is pulled in as much as possible, then, the area of connection of the abdominal muscles is fixed with the help of the operator, placing a hand in the middle of the abdomen: the elbow on the pubic bone, the wrist on the stomach area, and the abdomen is pressed; then, during breath holing, straightened legs are raised 30 cm from the surface, while raising the head, shoulders, shoulder-blades, tensing the abs and the muscles of the ribs, the abdomen is forcibly pressed with the operator's hand, not allowing the lower back to bend and the abdominal muscles to lift; with the knuckles of the operator's free hand, which is balled into fist, dynamic rubbing is carried out in the up and down direction along the abdominal muscles and the white line of the abdomen for a few seconds until the skin reddens; the cycle is repeated, alternating the impact on both sides, in the presence of contraindications, the strain is reduced by eliminating the raising of the legs and the upper body.
